(19) United States
(12) Patent Application Publication
(10) Pub. No.: US 2017/0130205 A1
(43) Pub. Date: May 11, 2017

(54) METHOD FOR PREPARING INDUCED PLURIPOTENT STEM CELLS

(71) Applicant: TAIPEI VETERANS GENERAL HOSPITAL, TAIPEI (TW)

(72) Inventor: SHIH-HWA CHIOU, TAIPEI (TW)

(21) Appl. No.: 14/949,841

(22) Filed: Nov. 23, 2015

Related U.S. Application Data
(60) Provisional application No. 62/082,842, filed on Nov. 21, 2014.

(51) Int. Cl. C12N 5/074; A61K 31/381; A61K 38/47; G01N 33/50

(52) U.S. Cl. CPC C12N 5/0696; G01N 33/5061; A61K 31/381; A61K 38/47; C12Y 302/01022; G01N 2800/52; C12N 2506/1307; G01N 2800/325; C12N 2501/60; C12N 2501/415; C12N 2501/727; C12N 2501/603; C12N 2501/605; C12N 2501/602; C12N 2501/604; C12N 2501/606; C12N 2501/608

(57) ABSTRACT

The present invention relates to a novel method for preparing induced pluripotent stem cells (iPSCs) by introducing four genes, Oct-4, Sox2, Klf4, and Glial, into somatic cells. The present invention also relates to the iPSCs produced by the aforementioned method. Also provided is a process of drug selection for a heritable genetic disease by use of the iPSCs produced by the aforementioned method. In particular, wherein the inherited disease is Fabry disease. The present invention also relates to a method for treating Fabry-associated myocardiopathy in a subject in need thereof, and a method for determining prognosis in a subject with Fabry-associated myocardiopathy.

### RELATED APPLICATIONS

This application claims the benefit of U.S. provisional application No. 62/082,842, filed Nov. 21, 2014 under 35 U.S.C. §119, the entire content of which is incorporated herein by reference.

### FIELD OF THE INVENTION

The present invention relates generally to a method for preparing induced pluripotent stem cells (iPSCs) from somatic cells, and the iPSC(s) obtained by the method. The present invention also relates to a process of drug selection for a heritable genetic disease. The present invention also relates to a method for treating Fabry-associated myocardiopathy in a subject in need thereof, and a method for determining prognosis in a subject with Fabry-associated myocardiopathy.

### BACKGROUND OF THE INVENTION

Recently, the induced pluripotent stem cell (iPSC) technology has demonstrated that somatic cells derived from living patients might generate patient- or disease-specific cells that are similar to natural pluripotent stem cells, providing a great potential for modeling disease phenotypes (Ferreira et al., How induced pluripotent stem cells are redefining personalized medicine. Gene. 2013; 520:1-6). Yamanaka successfully produced iPSCs by the transfection of the genes that had been identified as particularly important in embryonic stem cells (ESCs), and isolated four key pluripotency genes essential for the production of pluripotent stem cells: Oct-3/4, Sox2, c-Myc, and Klf4 (Takahashi and Yamanaka, 2006, Induction of pluripotent stem cells from mouse embryonic and adult fibroblast cultures by defined factors. Cell 126, 663-676.).

Yamanaka et al. provided the method for preparing an induced pluripotent stem cell (iPSC) by nuclear reprogramming of a somatic cell through introducing the genes Oct3/4, Klf4, c-Myc and Sox2 in U.S. Pat. No. 8,058,065 (filed on Jun. 9, 2009 and issued on Nov. 15, 2011). The method for preparing somatic cells by inducing differentiation of the iPSC as obtained was disclosed in U.S. Pat. No. 8,129,187 (filed on Feb. 18, 2010 and issued on Mar. 6, 2012). Yamanaka et al. also provided a method for preparing an induced pluripotent stem cell (iPSC) by introducing the genes encoding Oct3/4, Klf4 and Sox2 without c-Myc (U.S. Pat. No. 8,278,104, filed on Jun. 13, 2008 and issued on Oct. 2, 2012). Besides, Sakurada et al. provides a method and platform for drug discovery using two or more populations of isolated cells differentiated from the iPSCs comprising an exogenous Oct3/4, Sox2 and Klf4 genes (U.S. Pat. No. 8,257,941, filed on Jun. 12, 2009 and issued on Sep. 4, 2012). Trion provided a method for generating integration-free human induced pluripotent stem cells from blood cells using one or more DNA expression vectors encoding the reprogramming factors (a) Oct4, Sox2, Klf4, and c-Myc; (b) Oct4, Sox2, and Klf4; (c) Oct4, Sox2, Klf4, c-Myc, and Nanog; or (d) Oct 4, Sox2, Lin-28, and Nanog (U.S. Pat. No. 8,048,675, filed on May 12, 2010 and issued on Nov. 1, 2011).

Fabry disease (FD) is an X-linked recessive lysosomal storage disorder resulting from a deficiency of α-galactosidase A (GLA), which is encoded by the GLA gene (Eng et al., Fabry disease: Twenty-three mutations including sense and antisense cpg alterations and identification of a deletional hot-spot in the alpha-galactosidase a gene. Hum Mol Genet. 1994; 3:1795-1799). ENREF 1 GLA deficiency leads to a progressive lysosomal accumulation of glycosphingolipids, predominantly globotriaosylceramide (Gb3), in various organs, including heart, brain, and kidney. ENREF 2 Depending on which organs are involved, cardiomyopathy, acroparesthesia, hypohydrosis, angiokeratoma, corneal opacities (cornea verticillata), and impaired renal function may occur in FD (Desnick et al., Fabry disease, an under-recognized multisystemic disorder: Expert recommendations for diagnosis, management, and enzyme replacement therapy. Ann Intern Med. 2003; 138:338-346). To date, more than 400 GLA mutations have been reported (Human Gene Mutation Database; http://www.hgmd.org), and IVS4+919G>A is the most common (82-86%) GLA mutation detected in Taiwan (Chien et al., Fabry disease: Incidence of the common later-onset alpha-galactosidase a ivs4+919g-->a mutation in taiwanese newborns—superiority of DNA-based to enzyme-based newborn screening for common mutations. Mol Med. 2012; 18:780-784). Patients with the IVS4+919G>A mutation, which is associated with a late-onset phenotype, typically present cardiac abnormalities (including left ventricular hypertrophy, cardiac arrhythmia, and cardiomyopathy) rather than the typical symptoms of FD (Lin et al., High incidence of the cardiac variant of Fabry disease revealed by newborn screening in the taiwan chinese population. Circ Cardiovasc Genet. 2009; 2:450-456). Although the etiology of FD has been identified as an inherited glycosphingolipidase with deficient enzyme activity, the precise mechanism by which FD with a late-onset GLA IVS4+919G>A mutation contributes to cardiomyopathy remains uncertain.

Because of the limitations of clinical samples from cardiac biopsies or primary CM cultures, the pathogenesis of FD-associated cardiomyopathy and abnormalities associated with the IVS4+919G>A mutation are undetermined. The generation of cardiomyocytes (CMs) or cardiogenic lineages in vitro might be an ideal model for investigating the pathogenesis and progression of FD cardiomyopathy. Recently, patient iPSC-derived CMs have recapitulated various pathophysiological features such as long-QT syndrome (Itzhaki et al., Modeling the long qt syndrome with induced pluripotent stem cells. Nature. 2011; 471:225-229, Josowitz et al., Induced pluripotent stem cell-derived cardiomyocytes as models for genetic cardiovascular disorders. Curr Opin Cardiol. 2011; 26:223-229), arrhythmogenic right ventricular dysplasia (Kim et al., Studying arrhythmogenic right ventricular dysplasia with patient-specific ipscs. Nature. 2013; 494:105-110) and hypertrophy cardiomyopathy (Limphong et al., Modeling human protein aggregation cardiomyopathy using murine induced pluripotent stem cells. Stem Cells Transl Med. 2013; 2:161-166). However, it remained mostly unknown to use proteomic analysis based on the clinical samples of cardiac biopsy or culture of cardiomyocyte for developing the cardiac-specific biomarkers or therapeutic targets in FD-associated cardiomyopathy.

Fabry cardiomyopathy (FC) is known as the major highly prevalent FD-associated morbidity (Eng et al., Safety and efficacy of recombinant human alpha-galactosidase A—replacement therapy in Fabry's disease. N Engl J Med. 2001; 345(1) :9-16). Left ventricular hypertrophy (LVH), the most common presentation of FC as a result of the progressive intracellular accumulation of globotriaosylceramide (Gb3), is potentially alleviated by early enzyme replacement therapy (ERT) with GLA (Schiffmann et al., Infusion of alpha-galactosidase A reduces tissue globotriaosylceramide storage in patients with Fabry disease. Proc Natl Acad Sci U S A. 2000; 97(1):365-70). Furthermore, clinical trials have demonstrated that ERT can reduce the risk of major clinical events, remodel the left ventricle, improve cardiac function, and increase exercise tolerance (Yousef et al., Left ventricular hypertrophy in Fabry disease: a practical approach to diagnosis. Eur Heart J. 2013; 34(11):802-8.). However, disease progression still occurs in a minority of FC patients, particularly those with myocardial fibrosis after ERT (Weidemann et al., Long-term effects of enzyme replacement therapy on fabry cardiomyopathy: evidence for a better outcome with early treatment. Circulation. 2009; 119(4):524-9). LysoGb3 has been used as an FD-specific marker; however, certain reports have indicated that Gb3 and lysoGb3 might not be suitable biomarkers for monitoring the long-term progression and therapeutic outcome of FC after ERT, especially for FC patients with myocardial fibrosis (FC-MC) (Vedder et al., The Dutch Fabry cohort: diversity of clinical manifestations and Gb3 levels. J Inherit Metab Dis. 2007; 30(1):68-78; Whitfield et al., Monitoring enzyme replacement therapy in Fabry disease--role of urine globotriaosylceramide. J Inherit Metab Dis. 2005; 28(1):21-33; Liu et al., Globotriaosylsphingosine (lyso-Gb3) might not be a reliable marker for monitoring the long-term therapeutic outcomes of enzyme replacement therapy for late-onset Fabry patients with the Chinese hotspot mutation (IVS4+919G>A). Orphanet J Rare Dis. 2014; 9(1):111).

### BRIEF SUMMARY OF THE INVENTION

It is unexpectedly found in the present invention that induced pluripotent stem cells (iPSCs) could be successfully prepared by transfecting or transducing the transcription factor into isolated somatic cells, or contacting or exposing isolated somatic cells with/to transcription factor. In particular, the transfection factors are Oct-4, Sox2, Klf4, and Glial. In the present invention, it is also unexpectedly found that iPSCs were involved in a process of drug selection for a heritable genetic disease. It is unexpectedly found in the present invention that Alox 12/15 or 12(S)-HETE/15(S)-HETE can be used to identify whether a subject has poor prognosis for

Fabry-associated myocardiopathy. It is also unexpectedly found that an arachidonate lipoxygenases 12/15 (Alox 12/15) inhibitor is able to treat Fabry-associated myocardiopathy.

Accordingly, in one aspect, the present invention provides a method for preparing induced pluripotent stem cells (iPSCs) from somatic cells. The method comprises (a) transfecting or transducing the transcription factor into isolated somatic cells, or contacting or exposing isolated somatic cells with/to transcription factor, which the isolated somatic cells can express transcription factor; and (b) culturing the isolated somatic cells as obtained in step (a) under appropriate conditions, thereby converting the somatic cells into iPSCs and maintaining pluripotency and self-renewal ability, wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

In another aspect, the present invention provides a method for preparing iPSCs from somatic cells, wherein the isolated somatic cells are transfected or transduced with one or more plasmid or vector comprising transcription factor operably linked to a promoter, wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

In a further aspect, the present invention provides a method for preparing iPSCs from somatic cells, wherein the isolated somatic cells are fibroblasts, nerve cells, amniotic fluid cells, bone marrow cells, blood cells, myocardial cells, dermal or epidermal cells, connective tissue cells, chondrocytes, rod and cone cells, retinal pigment epithelia, or a pancreatic cells.

In a further yet aspect, the present invention provides a method for preparing iPSCs from somatic cells, wherein the iPSCs can differentiate to nervous system, teeth, hair, exocrine glands, epithelium, or mesenchyme from ectoderm, the muscle of smooth, cardiac and skeletal, the muscles of the tongue, the pharyngeal arches muscle, connective tissue, dermis and subcutaneous layer of the skin, bone and cartilage, dura mater, endothelium of blood vessels, red blood cells, white blood cells, microglia and Kupffer cells, the kidneys and the adrenal cortex cartilage, gonads, or keratinocytes from mesoderm, or lung cells, thyroid cells, pancreatic cells, liver cells, retinal pigment epithelia, or eyes from endoderm.

In another aspect, the invention provides a method for inducing the secretion of IP-10 which comprises administering to a subject in need thereof an effective amount of iPSCs or iPSC-CM.

In another aspect, the present invention provides an iPSC(s) obtained by the method in the present invention.

In still another aspect, further provided is a process of drug selection for a heritable genetic disease. The process comprises (1) isolating the somatic cells from a subject with a heritable genetic disease, (2) preparing the iPSCs as the method in the present invention, (3) differentiating the iPSCs obtained from step (2) into a cell line having the gene of the heritable genetic disease and affected by the disease, and (4) selecting a drug for improving the condition of cells of the cell line affected by the heritable genetic disease.

In an embodiment of the present invention, the heritable genetic disease is selected from the group consisting of Fabry disease, cystic fibrosis, sickle-cell anemia, polydactyly, Huntingdon's disease, ALA dehydratase deficiency, aceruloplasminemia, achondroplasia, Turner syndrome, Down syndrome, Klinefelter syndrome, Gaucher disease type 1 and type 2, Apert syndrome, Pfeiffer syndrome, acute intermittent porphyria, Canavan disease, Alzheimer's disease, Muenke syndrome.

In one more aspect, the present invention provides a method for treating Fabry-associated myocardiopathy in a subject in need thereof, comprising administering to the subject an effective amount of an arachidonate lipoxygenases 12/15 (Alox 12/15) inhibitor, wherein the Alox 12/15 inhibitor is selected from the group consisting of LOXBlock-1, LOXBlock-2 and LOXBlock-3 or a combination thereof. In one embodiment, the Alox 12/15 inhibitor is LOXBlock-1.

In another aspect, the present invention provides a method for determining whether a subject has poor prognosis for Fabry-associated myocardiopathy, comprising extracting a test sample of tissue from a subject; measuring the level of Alox 12/15 or 12(S)-HETE/15(S)-HETE in the test sample of tissue from the subject; and determining the prognosis of the subject, wherein an alteration of Alox 12/15 or 12(S)-HETE/15(S)-HETE level in the test sample, relative to the corresponding Alox 12/15 or 12(S)-HETE/15(S)-HETE level in a control sample of Fabry-disease free tissue, is indicative of the subject having a poor prognosis for Fabry-associated myocardiopathy.

In one yet aspect, the present invention provides a method for determining whether a subject has poor prognosis for Fabry-associated myocardiopathy, wherein the subject has received enzyme replacement therapy (ERT), comprising extracting a test sample of tissue from a subject; measuring the level of Alox 12/15 or 12(S)-HETE/15(S)-HETE in the test sample of tissue from the subject; and determining the prognosis of the subject, wherein a significant of Alox 12/15 or 12(S)-HETE/15(S)-HETE level in the test sample, relative to the corresponding Alox 12/15 or 12(S)-HETE/15(S)-HETE level in a control sample of Fabry-disease free tissue, is indicative of the subject having a poor prognosis for Fabry-associated myocardiopathy.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawing.

In the drawings:

FIG. 1A-1I show the characterization of FD patient with IVS4+919G>A mutation and generation of patient-specific iPSCs. FIG. 1A provides the image of transthoracic echocardiography demonstrated marked ventricular septum (VS) and posterior wall (PW) hypertrophy in LV (upper left). Histologic examination (H&E) staining of myocardium showed markedly hypertrophic and disarrayed myocytes with large perinuclear and sarcoplasmic vacuoles (upper right). Toluidine blue staining in myocardium proved glycosphingolipid accumulation (lower left). Scale bar, 250 μm. Electron microscopical examination of myocardium revealed lamellar bodies (Zebra body) that represented lysosomes containing glycolipid, TEM×60,000 (lower right). FIG. 1B provides the results of the sequence analysis confirming the existence of specific IVS4+919G>A mutation in the peripheral blood samples. FIG. 1C provides the DNA sequence showing the large 3445-bp deletion with breakpoints on introns 1 and 2, which contain multiple Alu repeat sequences, were detected in all ten patients. FIG. 1D shows the reprogramming protocol for FD-iPSCs (upper). Phase-contrast photomicrograph (lower left), ALP activity (lower middle) and embryoid body formation (lower right) of FD-iPSCs. Scale bar, 100 μm. FIG. 1E provides the image of the RT-PCR results that the FD-iPSCs expressed several human ESC marker genes, including Oct3/4, Sox2, Nanog, Rex1, DPPA2, DPPA4, DPPA5 and GDF3. FIG. 1F provides the image of the immunofluoresence indicating the pluripotency markers Oct3/4, Nanog, SSEA3, SSEA4, TRA-1-60 and TRA-1-81 in FD-iPSC clones. Scale bar, 100 μm. FIG. 1G provides the image of the immunofluoresence for SMA (mesodermal marker), NF (ectodermal marker) and AFP (endodermal marker) in each FD-iPSCs-derived differentiated cell, (upper). H&E staining for three germ layers including neuronal epithelium (ectoderm), cartilage and keratinocytes (mesoderm), and smooth muscle (mesoderm) of solid teratoma transplanted in the subrenal grafts of SCID mice (lower). Scale bar, 100 μm. FIG. 1H provides the image of chromosomal analysis indicated that the FD-iPSCs exhibiting a karyotype identical to that of the control iPSCs reprogrammed from normal dermal fibroblasts. FIG. 1I provides the results of the sequence analysis confirming the existence of specific IVS4+919G>A mutations in the FD-iPSC lines.

FIGS. 2A-2F show that FD-iPSC-CMs recapitulated FD-specific characteristic. FIG. 2A provides the image of the cardiac-specific differentiation protocol for FD-iPSC-CMs (upper). Phase-contrast photomicrograph of synchronized beating FD-iPSC-CMs (lower). FIG. 2B provides the images of immunofluoresence for cardiac markers α-Actinin and MYL2 in FD-iPSC-CMs at post-induction day 30. Scale bar, 15 μm. FIG. 2C provides the images of immunofluoresence for cardiac troponin T (cTnT) at post-induction day 60 (left) and the quantification of cell size for FD-iPSC-CMs (Pt1 to Pt3) at post-induction day 20, 30, 40 and 60 (right), demonstrating enlarged cellular size in FD-iPSC-CMs, compared with Ctrl-iPSC-CMs, after post-induction 30 days. Scale bar, 15 μm. FIG. 2D provides the quantitative RT-PCR showing the decreased GLA gene expression by cardio-specific induction in a time-dependent manner in FD-iPSC-CMs (Pt1 to Pt4). FIG. 2E provides the image of the Western blot indicating the time-dependent downregulation of GLA protein by cardio-specific induction in FD-iPSC-CMs (Pt1 to Pt3). FIG. 2F provides the results of the GLA enzyme assay showing the defect of GLA enzyme activity in FD-iPSC-CMs (Pt1 to Pt4). *P<0.05 vs. corresponding cells at D20.

FIG. 3 provides the images of the ultramicrostructural abnormalities and Gb3 accumulation in FD-iPSC-CMs. Electron microscopical examination of hypertrophic like FD-iPSC-CMs showed the deficiency of GLA resulted pathophysiological characteristics of FD cardiomyopathy, including morphological abnormalities (organelle and/or cytoplasm loss) and Gb3 accumulation (appear within enlarged secondary lysosomes as lamellated membrane structures, called zebra bodies) (TEM×8,000).

FIGS. 4A-4F show the enlarged cell size and impaired electrophysiology in FD-iPSC-CMs. FIG. 4A provides the results of the microarray analysis of Ctrl-iPSC-CMs and FD-iPSC-CM at post-induction 0, 20, 30, 40, and 60, showing the profiles of the differentially-expressed genes based on their functions in the Gene Ontology database. FIG. 4B-provides the quantitative RT-PCR indicating the upregulation of cardiac hypertrophy-associated genes ANF, ACT1C1, MYL2, MYL7 in FD-iPSC-CMs. FIG. 4C provides the measurement of the field potentials of contractile iPSC-CMs by the MED 64 system. FIG. 4D shows the representative tracings of the isoproterenol for Ctrl-iPSC-CMs and FD-iPSC-CMs. FIG. 4E provides the quantification showing the dose-dependent effect of isoproterenol (0, 1, 10 μM) on the field potentials of ontractile Ctrl-iPSC-CMs and FD-iPSC-CMs at post-induction day 60. FIG. 4F provides the quantification showing the dose-dependent effect of verapamil (0, 10, 100 nM) on the field potentials of ontractile Ctrl-iPSC-CMs and FD-iPSC-CMs at post-induction day 60. *P<0.05 vs.Ctrl-iPSC-CM treated with corresponding dose.

FIGS. 5A-5F show the upregulation of Alox12/Alox15 in FD-iPSC-CMs. FIG. 5A provides the schematic diagram of the MS-based proteomics, showing how candidate markers were identified in FD-iPSC-CMs. FIG. 5B provides the pie chart showing the GO classification of gene functions in fractionated proteins from FD-iPSC-CMs. FIG. 5C provides the results of the proteome screening found at least significant 14 candidate markers in FD-iPSC-CMs. FIG. 5D provides the image of the Western blotting confirming the upregulation of proteome screening-found candidate markers Alox12 and Alox15 in FD-iPSC-CMs. FIG. 5E provides the image of the immunofluorescence, and FIG. 5F shows the quantification confirming the upregulation of proteome screening-found candidate markers Alox12 and Alox15 in FD-iPSC-CMs. Scale bar, 15 μm. *P<0.05 vs.Ctrl-iPSC-CM.

FIG. 6 shows the metabolites of Alox12 and Alox15, and the secretion of 12-HETE and 15-HETE in a time-dependent manner (days 30 to 60).

FIGS. 7A-7F show the Gb3 stimulated Alox12/Alox15 expression and 12/15-HETE secretion. FIG. 7A provides the images of the Western blotting revealing that Gb3 stimulated Alox12/Alox15 protein expression in ESC-CMs, Ctrl-iPSC-CMs and FD-iPSC-CMs (Pt1, Pt2, Pt3 and Pt4) at post-induction day 20. FIG. 7B provides the images of the Western blotting revealing that Gb3 stimulated Alox12/Alox15 protein expression in ESC-CMs, Ctrl-iPSC-CMs and FD-iPSC-CMs (Pt5, Pt6, Pt7 and Pt8) at post-induction day 20. FIG. 7C provides the image of the immunofluoresence revealing the upregulation of Alox12 in FD-iPSC-CMs upon Gb3 treatment (15 μmole/L; 24 hours). FIG. 7D provides the image of the immunofluoresence revealing the upregulation of Alox15. Scale bar, XXX μm. FIG. 7E provides the results of the ELISA indicating the 12-HETE secretion from FD-iPSC-CMs (Pt1 to Pt8) upon Gb3 treatment (15 μmole/L; 24 hours). FIG. 7F provides the results of the ELISA indicating the 15-HETE secretion from FD-iPSC-CMs (Pt1 to Pt8); *P<0.05 vs. corresponding cells without Gb3 treatment.

FIGS. 8A-8E shows that Alox12/Alox15 as potential markers in FD patients with IVS4+919G>A mutation. FIG. 8A provides the images of TEM showing the typical lysosomal abnormalities (Membrane-bound lamellar myelin bodies in cardiac biopsy samples from FD patients with IVS4+919G>A mutation (left: TEM×30,000; right: TEM×60,000). FIG. 8B provides the images of the immunohistochemistry staining; and FIG. 8C shows the quantification revealing the upregulation of Alox12 and 15 in heart biopsy samples from FD patients (Pt1, Pt2) with IVS4+919G>A mutation. Scale bar, 100 μm. FIG. 8D shows the high 12-HETE levels in the serum samples from FD patients with IVS4+919G>A mutation and FIG. 8F shows the high 15-HETE levels. *P<0.05 vs. Ctrl.

FIGS. 9A-9B show the schemes for the identification of potential FD-specific markers and the relationship between deficiency of GLA and upregulation of Alox12/15. FIG. 9A shows the schemes of patient-specific somatic cells (heart/skin biopsy and blood collection) are confirmed with IVS4+919G>A mutation, reprogrammed to pluripotency by OSKG factors, differentiated to cardiac lineages, analyzed by LC/MS-based proteomics, and searching novel FD-specific markers for diagnosis/prediction of FD. FIG. 9B shows the schematic model for the relationship between deficiency of GLA and upregulation of Alox12/15. (left) Normal state: CMs retain GLA activity, no Gb3 accumulation is observed. (right) carried IVS4+919G>A mutation: CMs lose GLA activity, Gb3 accumulation, induce hypotrophy inflammatory response (Alox12/15 upregulation), 12/15-HETE secretion increase.

FIGS. 10A-10J shows the generation of patient-specific FC-iPSC-CMs. FIG. 10A shows that transthoracic echocardiography demonstrated marked ventricular septum (VS) and posterior wall (PW) hypertrophy of the LV (upper left); and a histological examination (hematoxylin and eosin (H&E) staining) of the myocardium revealed markedly hypertrophic and disorganized myocytes with large perinuclear and sarcoplasmic vacuoles (upper right). Toluidine blue staining of the myocardium verified the accumulation of glycosphingolipids (lower left). The scale bar is 250 μm. Transmission electron microscopic examination of the myocardium revealed lamellar bodies (zebra bodies) representing lysosomes containing glycolipids, ×60,000 (lower right). FIG. 10B shows the results of the sequence analysis confirming the existence of the specific IVS4+919G>A mutation in the peripheral blood samples. FIG. 10C shows the scheme for generating patient-specific FC-iPSC-CMs from blood samples. FIG. 10D shows the phase-contrast photomicrograph (left), ALP activity (middle) and embryoid body formation (right) in FC-iPSCs. The scale bar is 100 μm. FIG. 10E shows the immunofluorescence for cardiac markers [symbol] α-actinin and MYL2 in FC-iPSC-CMs at post-differentiation day 14. The scale bar is 15 μm. FIG. 10F shows that sequence analysis confirmed the existence of the specific IVS4+919G>A mutation in patient-specific FC-iPSC-CMs. FIG. 10G shows the immunofluorescence for cardiac troponin T (cTnT) at post-induction day 60 (left) and the quantification of FC-iPSC-CMs size at post-induction days 20, 30, 40 and 60 (right). The scale bar is 15 μm. FIG. 10H shows the mRNA levels of GLA in patient-derived FD-iPSC-CM at the indicated time points after the induction of differentiation. FIG. 10I provides the image of the Western blot analysis of GLA protein levels in differentiated FC-iPSC-CM derived from patient 1. FIG. 10J shows the evaluation of GLA activity in patient-derived FC-iPSC-CM at indicated post-differentiation time point. Data shown panels 1G, 1H, and IJ are the mean±SD of three independent experiments. In panel 1G, *P<0.05 vs. Ctrl-iPSC-CM. In panels 1H and 1J, *P<0.05 vs. D20 from corresponding patient.

FIG. 11 shows the ultramicrostructural abnormalities and Gb3 accumulation in FD-iPSC-CMs from day 20 to day 60 post-differentiation. Electron microscopical examination of hypertrophic like FD-iPSC-CMs showed the deficiency of α-Gal A resulted in pathophysiological characteristics of FD cardiomyopathy, including morphological abnormalities (organelle and/or cytoplasm loss) and Gb3 accumulation (appear within enlarged secondary lysosomes as lamellated membrane structures, called zebra bodies) (TEM×10,000).

FIG. 12A-12H show that Alox12/15 and its secretory metabolites 12(S)-HETE/15(S)-HETE are the most upregulated factors in FC-iPSC-CMs and clinical FC patients. FIG. 12A shows the schematic diagram depicting the screening for suitable biomarkers using LC/MS and the FC-iPSC-CM platform derived from FC patient blood. FIG. 12B provides the images of the Western blotting and FIG. 12C shows the immunofluorescence results, confirming the upregulation of Alox12/15 in FC-iPSC-CMs in comparison to Ctrl-iPSC-CMs (*p<0.05). The scale bar is 15 μm. FIG. 12D shows the ELISA analysis of the secreted levels of 12(S)-HETE, and FIG. 12E shows 15(S)-HETE in conditioned medium from the cultures of FC-iPSC-CMs, compared with control medium from the cultures of Ctrl-iPSC-CMs and ESC-CMs. FIG. 12F shows a representative electron microscopic image showing typical morphological abnormalities in the heart biopsy samples from FC patients. FIG. 12 G shows that immunohistochemistry staining revealing the upregulation of Alox12/15 in the heart biopsy samples from FC patients (Pt 1 and Pt 2) vs. control biopsy samples. The scale bar is 100 μm. FIG. 12H shows the serum levels of 12(S)-HETE and 15(S)-HETE in the samples from 32 patients initially diagnosed as FC and 30 normal patient controls. Data shown in FIGS. 12D and 12E are the mean±SD of three independent experiments. In FIGS. 12D, 12E and 12G, *P<0.05 vs. Ctrl-iPSC-CM from corresponding patient.

FIG. 13A-13H show the regulation of fibrosis-associated genes and Alox12/15 in the late differentiated stage of FC-iPSC-CMs. FIG. 13A shows the mRNA expression levels of TGFβ, MMP1, and collagen 1 in FC-iPSC-CMs and Ctrl-iPSC-CM at indicated time points after the induction of myocardial differentiation. FIG. 13B shows GLA enzyme activity in various lines of Ctrl-iPSC-CMs and FC-iPSC-CMs with or without the administration of GLA. FIG. 13C shows the scheme depicting the experimental design of early-administration and FIG. 13D shows the late-administration of ERT in the FC-iPSC-CM platform. FIG. 13E shows the immunohistochemistry analysis at day 60 post-differentiation of the expression ratio of Alox12/15, MMP1, CollagenI, and TGF-β in FC-iPSC-CMs that received early-administration (from day 20 to day 60 post-induction) and FIG. 13F shows the late-administration (from day 40 to day 60 post-differentiation) of GLA. Bar size: 15 μM. FIG. 13 G shows the time-course analysis of the relative size of FC-iPSC-CM cells receiving early-administration and FIG. 13 H shows the late-administration of GLA or vehicle. Data shown panels 13A, 13B, 13G and 13H are the mean±SD of three independent experiments. In FIG. 13 A, *P<0.05 vs. Ctrl-iPSC-CM. In FIGS. 13G and 13H, *P<0.05 vs. D20 from corresponding patient.

FIG. 14A-14F show the secreted levels of LysoGb3, 12(S)-HETE, and 15(S)-HETE from FC-iPSC-CMs with the early- and late-administration of GLA. FIG. 14A shows the time-course evaluation of LysoGb3. FIG. 14B shows 12(S)-HETE, and FIG. 14C shows 15(S)-HETE in FC-iPSC-CMs receiving early-administration of GLA or vehicle during the 60 days of myocardial differentiation. FIG. 14D shows the time-course evaluation of LysoGb3. FIG. 14E shows 12(S)-HETE, and FIG. 14F shows 15(S)-HETE in FC-iPSC-CMs receiving late-administration of GLA or vehicle during the 60 days of myocardial differentiation. Data are the mean±SD of six independent experiments. In FIG. 14A through 14F, *P<0.05 vs. Untreated.

FIG. 15A-15D show the effect of administration of GLA at early or late onset on Alox12/15 expression and cardiomyocyte size. FIG. 15A shows that cTnT-positive FC-iPSC-CMs was quantified to evaluate the cell size of FC-iPSC-CM receiving late-administration of either GLA alone. FIG. 15B shows a combination of GLA and LOXBlock-1. FIG. 15 C shows the effect of late administration of GLA or a combination of GLA and LOXBlock-1 on the secretion of 12(S)-HETE, 15(S)-HETE and LysoGb3, and FIG. 15D shows the relative expression ratio of MMP1, TGBb, and collagen-1, in FC-iPSC-CM.

FIG. 16A-16F show the immunohistochemistry staining of Alox12/15, TGFβ and MMP1 in myocardial biopsy samples from ERT-treated FC patients with myocardial fibrosis. FIG. 16A shows the identification of myocardial fibrosis in FC patients using CMR-LGE. FIG. 16B shows that the consecutive CMR-LGE scanning in FC patient that expressed no fibrosis sign initially (left) but appeared myocardial-fibrosis in the 2^{nd} CMR-LGE (right). FIG. 16C shows the immunohistochemistry staining of Alox12/15, TGF-β and MMP1 in myocardial biopsy samples from ERT-treated FC patients with or without myocardial fibrosis. FIG. 16D shows the positive stains of Alox12/15, and FIG. 16E shows the positive stains of TGFβ and FIG. 16F shows the positive stains of MMP1, which were quantified and showed as relative ratio to the controls. *P<0.05 vs. Ctrl. Fibrosis Group, n=4. Non-fibrosis Group, n=7.

FIG. 17A-17G show the negative correlation between 12(S)-HETE/15(S)-HETE and systolic functions in FC patients with myocardial fibrosis. FIG. 17A shows the linear regression between the changes in the left ventricular ejection fraction (LVEF) and changes in the levels of 12(S)-HETE in all 17 FC patients with myocardial fibrosis despite ERT, FIG. 17B shows that of 15(S)-HETE and FIG. 17C shows that of lysoGb3. FIG. 17D shows the changes of the levels of LVEF, FIG. 17E shows that of 12(S)-HETE, FIG. 17F shows that of 15(S)-HETE, shown in FIG. 17 F, and FIG. 17G shows that of lysoGb3, between initial and follow-up tests in the 7 FC patients that expressed no fibrosis sign in the 1^{st} CMR-LGE scan but appear to be fibrosis in the 2^{nd} CMR-LGE.

### DETAILED DESCRIPTION OF THE INVENTION

Unless defined otherwise, all technical and scientific terms used herein have the same meaning as commonly understood by a person skilled in the art to which this invention belongs. All publications mentioned herein are incorporated herein by reference to disclose and describe the methods and/or materials in connection with which the publications are cited.

As used herein, the article “a” or “an” means one or more than one (that is, at least one) of the grammatical object of the article, unless otherwise made clear in the specific use of the article in only a singular sense.

The term “pluripotent” as used herein refers to a cell with the capacity, under different conditions, to differentiate to more than one differentiated cell type, and preferably to differentiate to cell types characteristic of all three germ cell layers.

As used herein, the terms “iPSCs” and “induced pluripotent stem cells” are used interchangeably and refers to a pluripotent stem cell artificially derived (e.g., induced or by complete reversal) from a non-pluripotent cell, typically an adult somatic cell, for example, by inducing a forced expression of one or more genes, and are capable of self-renewal and differentiation into several different cell types after proper induction.

As used herein, the term “subject” refers to a human or a mammal, such as a patient, a companion animal (e.g., dog, cat, and the like), a farm animal (e.g., cow, sheep, pig, horse, and the like) or a laboratory animal (e.g., rat, mouse, guinea pig, and the like).

As used herein, the term “adult cell” refers to a cell found throughout the body after embryonic development.

The term “reprogramming” used herein refers to a process of erasure and remodeling of epigenetic marks, such as DNA methylation wherein the original DNA methylation patterns are erased and re-established.

The term “somatic cell” as used herein refers to any cell forming the body of an organism that are not germ line cells (e. g. sperm and ova, the cells from which they are made (gametocytes)) and undifferentiated stem cells. Internal organs, skin, bones, blood and connective tissue are all made up of somatic cells. The somatic cell including but not limited to fibroblasts, nerve cells, amniotic fluid cells, bone marrow cells, blood cells, myocardial cells, dermal or epidermal cells, connective tissue cells, chondrocytes, rod and cone cells, retinal pigment epithelia, or pancreatic cells. Preferred somatic cells used in the method described herein are dermal fibroblasts or peripheral blood mononuclear cells.

The term “transfect” as used herein refers to the process of introducing nucleic acids into a host cell by any method, without the use of a virus or viral particle carrier.

The term “transduce” refers to the viral transfer of genetic material and its expression in a host cell.

The term “transcription factor” refers to a protein that regulates expression of one or more genes involved in cell. The transcription factor including but not limited to Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

The term “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes vehicles for introducing the nucleic acid into a cell in vitro, ex vivo or in vivo. A large number of vectors known in the art may be used to manipulate nucleic acids, incorporate response elements and promoters into genes, etc.

The term “differentiation”, as used herein, refers to a phenomenon in which the structure or function of cells is specialized during the division, proliferation and growth thereof. Induced pluripotent stem cells give rise to progenitor cells that gradually differentiate into committed cell lineages (e.g., ectodermal, mesodermal, and endodermal cells, etc.), and may further differentiate into other types of progenitor cells (e.g., hemangioblast, etc.), which in turn generate terminally differentiated cell types (e.g., vascular endothelial cells and vascular smooth muscle cells, etc.) that have specialized functions in the specialized tissues (e.g., blood vessels, etc.).

The term “ectoderm” refers to the outermost germ layer of cells derived from the inner cell mass of the blastocyst. Through cell division and specialization, the ectoderm gives rise to the cells including but not limited to the nervous system (spine, peripheral nerves and brain), sensory organs, skin (e.g. epithelia), exocrine glands, mesenchyme, and related structures (e.g. sweat glands, teeth, hair, and nails).

The term “mesoderm” as used herein, refers to the germ layer that can forms but not limited to muscles, the heart, the circulatory and excretory systems, and the dermis, skeleton, and other supportive and connective tissue. It also gives rise to the notochord, a supporting structure between the neural canal and the primitive gut. In many animals, including vertebrates, the mesoderm surrounds a cavity known as the coelom, the space that contains the viscera.

The term “endoderm,” as used herein, refers to the innermost germ layer of the early embryo. It gives rise but not limited to the entire alimentary canal except part of the mouth, pharynx and the terminal part of the rectum (which are lined by involutions of the ectoderm), the lining cells of all the glands which open into the digestive tube, including those of the liver and pancreas; the trachea, bronchi, and alveoli of the lungs; the lining of the follicles of the thyroid gland and thymus; the epithelia of the auditory tube and tympanic cavity; the urinary bladder and part of the urethra.

As used herein, the term “genetic disease or disorder” refers to an illness caused by one or more abnormalities in the genome, especially a condition that is present from birth (congenital). Most genetic disorders may or may not be heritable, i.e., passed down from the parents' genes. In non-heritable genetic diseases or disorders, defects may be caused by new mutations or changes to the DNA. In heritable cases, the mutations or changes would occur in the germ lines. The heritable genetic diseases include but are not limited to Fabry disease, cystic fibrosis, sickle-cell anemia, polydactyly, Huntingdon's disease, ALA dehydratase deficiency, aceruloplasminemia, achondroplasia, Turner syndrome, Down syndrome, Klinefelter syndrome, Gaucher disease type 1 and type 2, Apert syndrome, Pfeiffer syndrome, acute intermittent porphyria, Canavan disease, Alzheimer's disease, and Muenke syndrome.

In one aspect, the present invention provides a method for preparing induced pluripotent stem cells (iPSCs) from somatic cells, comprising: (a) transfecting or transducing the transcription factor into isolated somatic cells, or contacting or exposing isolated somatic cells with/to transcription factor, which the isolated somatic cells can express transcription factor; and; and (b) culturing the isolated somatic cells as obtained in step (a) under appropriate conditions, thereby converting the somatic cells into iPSCs and maintaining pluripotency and self-renewal ability, wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

In another aspect, the present invention provides a method for preparing induced pluripotent stem cell (iPSCs) from somatic cells, wherein the isolated somatic cells are transfected or transduced with one or more plasmid or vector comprising transcription factor operably linked to a promoter, wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof. The transfection or transduction of the isolated somatic cells may be performed according to any of the methods known by those skilled in the art. In one embodiment of the invention, wherein the isolated somatic cells are transfected by electroporation. In one another embodiment of the invention, the isolated somatic cells can be transfected or transduced with one or more plasmid or viral vectors comprising Oct-4, Sox2, Klf4, and Glial operably linked to a promoter.

In one another aspect, the present invention provides a method for preparing induced pluripotent stem cells (iPSCs) from somatic cells, wherein the isolated somatic cells are fibroblasts, nerve cells, amniotic fluid cells, bone marrow cells, blood cells, myocardial cells, dermal or epidermal cells, connective tissue cells, chondrocytes, rod and cone cells, retinal pigment epithelia, or pancreatic cells In one embodiment of the invention, wherein the fibroblasts are dermal fibroblasts. In another specific embodiment, wherein the blood cells are peripheral blood mononuclear cells.

In one aspect, the present invention provides a method for preparing iPSCs from somatic cells, wherein the iPSCs can differentiate to nervous system, teeth, hair, exocrine glands, epithelia, or mesenchyme from ectoderm, the muscle of smooth, cardiac and skeletal, the muscles of the tongue, the pharyngeal arches muscle, connective tissue, dermis and subcutaneous layer of the skin, bone and cartilage, dura mater, endothelium of blood vessels, red blood cells, white blood cells, microglia and Kupffer cells, the kidneys and the adrenal cortex cartilage, gonads, or keratinocytes from mesoderm, or lung cells, thyroid cells, pancreatic cells, liver cells, retinal pigment epithelium, or eyes from endoderm.

Also provided in this invention is an iPSC produced by the method described herein.

Also provided in the invention is a process of drug selection for the treatment of a heritable genetic disease, comprising the steps of: (1) isolating the somatic cells from a subject with a heritable genetic disease, (2) preparing the iPSCs as the method described herein, (3) differentiating the iPSCs obtained from step (2) into a cell line having the gene of the heritable genetic disease and affected by the disease, and (4) selecting a drug for improving the condition of the cell line affected by the heritable genetic disease. In the embodiments of the invention, the heritable genetic disease is selected from the group consisting of Fabry disease, cystic fibrosis, sickle-cell anemia, polydactyly, Huntingdon's disease, ALA dehydratase deficiency, aceruloplasminemia, achondroplasia, Turner syndrome, Down syndrome, Klinefelter syndrome,

Gaucher disease type 1 and type 2, Apert syndrome, Pfeiffer syndrome, acute intermittent porphyria, Canavan disease, Alzheimer's disease, and Muenke syndrome.

The present invention also provides a process of drug selection for the treatment of Fabry disease, comprising the steps of: (1) isolating the somatic cells from a subject with Fabry disease, (2) preparing the iPSCs as the method described herein, (3) differentiating the iPSCs obtained from step (2) into hypertrophic cardiomyocytes having Fabry disease and affected by the disease, and (4) selecting a drug for improving the condition of hypertrophic cardiomyocytes affected by Fabry disease. In one specific embodiment, wherein the hypertrophic cardiomyocytes detected by one or more biomarkers selected from the group consisting of Alox12, Alox15, 12-HETE, and 15-HETE, which have a high level in one or more of the biomarkers as compared to normal cardiomyocytes.

In another aspect, the present invention provides a method for treating Fabry-associated myocardiopathy in a subject in need thereof, comprising administering to the subject an effective amount of an arachidonate lipoxygenases 12/15 (Alox 12/15) inhibitor, wherein the Alox 12/15 inhibitor is selected from the group consisting of LOXBlock-1, LOXBlock-2 and LOXBlock-3 or a combination thereof.

In a further aspect, the present invention provides a method for treating Fabry-associated myocardiopathy in a subject in need thereof, comprising administering to the subject an effective amount of an arachidonate lipoxygenases 12/15 (Alox 12/15) inhibitor, wherein the Alox 12/15 inhibitor is administered simultaneously with an effective amount of α-galactosidase A (GLA).

In another aspect, the present invention provides a method for determining whether a subject has poor prognosis for Fabry-associated myocardiopathy, comprising extracting a test sample of tissue from a subject; measuring the level of Alox 12/15 or 12(S)-HETE/15(S)-HETE in the test sample of tissue from the subject; and determine the prognosis of the subject, wherein an alteration in the levels of Alox 12/15 or 12(S)-HETE/15(S)-HETE level in the test sample, relative to the corresponding Alox 12/15 or 12(S)-HETE/15(S)-HETE level in a control sample of Fabry-disease free tissue, is indicative of the subject having a poor prognosis for Fabry-associated myocardiopathy. In one embodiment of the invention, the subject has received enzyme replacement therapy (ERT). In another embodiment of the invention, the poor prognosis for Fabry-associated myocardipathy is manifested as a syndrome selected from the group consisting of myocardial fibrosis and decreased systolic left ventricular (LV) function or a combination thereof.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated herein by reference in their entirety.

### EXAMPLES

### I

### I. Materials and Methods

1. Generation of Patient-Specific iPSCs

The study followed the tenets of the Declaration of Helsinki, and the protocols for this study were approved by the board of Taipei Veterans General Hospital under No. 2013-06-025B. The samples were obtained after the patients gave informed consent. Dermal fibroblasts were isolated from the patients with Fabry diseases (FD) by punch biopsy. Briefly, the iPSCs were reprogrammed by the transduction of retroviral vectors encoding four transcription factors, Oct-4, Sox2, Klf4, and Glisl, as described previously (Maekawa et al., Direct reprogramming of somatic cells is promoted by maternal transcription factor glisl. Nature. 2011; 474:225-229). Plat-A cells were incubated overnight at a density of 2.5×10⁶ cells per 100-mm dish. The next day, 10 μg of pMX-containing cDNA was transfected into the Plat-A cells with 10 ml of fresh DMEM using TransIT®-LT1 (Mirus, Madison, Wis., USA). At 48 hours after transfection, the virus-containing medium was collected for target cell infection. Prior to viral infection, 5×10⁴ target cells were seeded per well into 6-well plates 1 day prior to the transduction. Supernatants containing equal amounts of each of the 4 retroviruses were filtered through a 0.45-pm filter and supplemented with 10 μg/ml of polybrene (Sigma), and the medium in the 6-well plates was replaced with the virus-containing medium. The 6-well plates were centrifuged at 2,250 rpm for 1 hour, and the medium was replaced. At day 7 post-infection, the target cells were passaged onto mitotically inactivated MEF feeder layers and cultured using human embryonic stem cell (ESC) medium. SB431542 (2 μM, Stemgent), PD0325901 (0.5 μM, Stemgent), and thiazovivin (0.5 μM) were added to the culture medium to aid colony formation. The drug-containing medium was replaced daily until iPSC colonies were detected. The undifferentiated iPSCs were maintained on mitotically inactivated MEFs (50,000 cells/cm²) in human ESC medium (DMEM/F12 (Gibco), supplemented with 20% KnockOut serum replacer (KSR; Invitrogen), 0.1 mM of non-essential amino acids (Invitrogen), 1 mM of L-glutamine, 0.1 mM of β-mercaptoethanol, 10 ng/ml of recombinant human basic fibroblast growth factor (bFGF), and antibiotics (Gibco). To prevent cell contamination by MEFs, these iPSCs were transferred to a feeder-free/serum-free culture in HESF V2 medium (Cell Science & Technology Institute, Inc.) without KSR supplementation. The characterization and establishment of induced pluripotent stem cell (iPSC) were shown in Table 1.

TABLE 1
Characterization and establishment of induced pluripotent stem cell (iPSC) derived from 10 FD patients with
IVS4 + 919G > A mutation

| | Patient | Patient | Patient | Patient | Patient | Patient | Patient | Patient | Patient | Patient |
|---|---|---|---|---|---|---|---|---|---|---|
| | 1 | 2 | 3 | 4 | 5 | 6 | 7 | 8 | 9 | 10 |
| Patient Information | | | | | | | | | | |
| Sex (F/M)*/age | F/60 | M/67 | M/48 | M/65 | M/45 | M/49 | M/65 | M/74 | F/40 | F/61 |
| (year-old) | | | | | | | | | | |
| Origin Samples | SB** | SB | SB | SB | SB | SB | SB | SB | SB | SB |
| Reprogramming | 0.2% | 0.3% | 0.05% | 0.4% | 0.2% | 0.6% | 0.3% | 0.5% | 0.5% | 0.1% |
| Efficiency (OSKG) | | | | | | | | | | |
| Cell passages of | >50 | >50 | >50 | >50 | >30 | >30 | >30 | >30 | >30 | >30 |
| iPSC | | | | | | | | | | |
| FD-iPSC | | | | | | | | | | |
| characterization | | | | | | | | | | |
| Alkaline phosphatase | + | + | + | + | + | + | + | + | + | + |
| (ALP) | | | | | | | | | | |
| qPCR (Stemness | + | + | + | + | + | + | + | + | + | + |
| genes) | | | | | | | | | | |
| Immunofluorescence | + | + | + | + | + | + | + | + | + | + |
| (Oct4, Nanog, | | | | | | | | | | |
| SSEA3, SSEA4, | | | | | | | | | | |
| Tra 1-81, Tra 1-60) | | | | | | | | | | |
| Karyotype | normal/ | normal/ | normal/ | normal/ | normal/ | normal/ | normal/ | normal/ | normal/ | normal/ |
| | stable | stable | stable | stable | stable | stable | stable | stable | stable | stable |
| Tridermal | + | + | + | + | + | + | + | + | + | + |
| Differentiation (in | | | | | | | | | | |
| vitro) | | | | | | | | | | |
| Teratomas formation | + | + | + | + | + | + | + | + | + | + |
| (in vitro) | | | | | | | | | | |
| IVS4 + 919G > A | + | + | + | + | + | + | + | + | + | + |
| mutatoin | | | | | | | | | | |

*F: female, M: male.
**SB: skin biopsy for culturing fibroblasts. More than 30: passage: iPSCs remained stable through 30 passages and showed ESC-like pluripotent property.

2. In Vitro Differentiation of iPSCs

The iPSCs were dispersed into small clumps using dispase (Sigma-Aldrich, Mo., USA; 1 mg/ml for 30 min) and transferred onto ultra-low attachment plates (Corning, N.Y., USA) for embryoid body formation. After 3 days, the aggregated cells were plated onto 0.1% gelatin-coated culture dishes with the FBS-containing medium. The medium was changed every 2 days. The cells were stained with an anti-α-smooth muscle actin monoclonal antibody (04-1094, Millipore), an anti-NF antibody (N1501, Dako), and an anti-alpha-fetoprotein monoclonal antibody (3903, Cell Signaling).

### 3. Teratoma Formation and Histological Analysis

Undifferentiated iPSCs (1×10⁶) were suspended in phosphate-buffered saline (PBS) and delivered by a 25-gauge syringe (BD Biosciences) to the subrenal capsule of 10- week old NOD SCID mice (BioLASCO). Eight weeks after the injection, tumors were dissected from the mice. The samples were weighed, fixed in PBS containing 4% formaldehyde, and embedded in paraffin. The sections were stained with hematoxylin and eosin.

### 4. Quantitative PCR and RT-PCR for the Marker Genes

Reverse transcription reactions were performed using SuperScript III reverse transcription (Invitrogen). The resulting cDNA was used for quantitative PCR (qPCR) and RT-PCR. qPCR was performed with Power SYBR Green PCR Master Mix (Applied Biosystems) according to the manufacturer's instructions. The signals were detected with a 7900HT Fast Real-Time PCR system (Applied Biosystems). The primer sequences are listed in Table 2.

TABLE 2
The sequences of the primers for quantitative RT-PCR

| Gene | | Product | Tm (° C.) |
|---|---|---|---|
| (Accession No.) | Primer Sequence | (5′ to 3′) | size (bp) |
| hOCT4 | F: CTTCAGGCACTGTGTTCATTG | 672 | 60 |
| NM_001159542.1 | R: TTTGGCTGAACACCTTCCCA | | |
| hSOX2 | F: GCCCTGCAGTACAACTCCAT | 735 | 60 |
| NM_003106.3 | R: TTCCTGCAAAGCTCCTACCG | | |
| hNANOG | F: GAAGACAAGGTCCCGGTCAA | 709 | 60 |
| NM_024865.2 | R: GGATTCAGCCAGTGTCCAGA | | |
| REX1 | F: GTGGGCCTTATGTGATGGCT | 759 | 60 |
| NM_174900.3 | R: TGCGTTAGGATGTGGGCTTT | | |
| hDPPA2 | F: CCGTCCCCGCAATCTCCTTCCATC | 606 | 60 |
| NM_138815.3 | R: ATGATGCCAACATGGCTCCCGGTG | | |
| hDppa4 | F: TAGCACAGCAAAAGAGGCCA | 635 | 60 |
| NM_018189.3 | R: TGCATGGCCCATAAACAGGT | | |
| hDppa5 | F: CGGCACGTAGACATATCCCG | 366 | 60 |
| NM_001025290.2 | R: GGCTTCATTGCATTGGCTGG | | |
| hGDF3 | F: GTTTGTGTTGCGGTCAGTCC | 501 | 60 |
| NM_020634.1 | R: TTGGTGGGGATACACACAGC | | |
| hGAPDH | F: GTCGCCAGCCGAGCCACATC | 83 | 60 |
| NM_002046.5 | R: CCAGGCGCCCAATACGACCA | | |
| hANF | F: GCCTAGGGACAGACTGCAAG | 170 | 60 |
| NM_006172.3 | R: GGCGAGGAAGTCACCATCAA | | |
| hACTA1 | F: CAGGGCGTCATGGTCGGT | 141 | 60 |
| NM_001100.3 | R: GTGCCAGATCTTCTCCATGTCATC | | |
| hMYL2 | F: CTCATCTCTCTCCCCCGAGT | 161 | 60 |
| NM_000432.3 | R: TGGAACATGGCCTCTGGATG | | |
| hMYL7 | F: GGAGTTCAAAGAAGCCTTCAGC | 112 | 60 |
| NM_021223.2 | R: CTCCTCTGGGACACTCACCT | | |

5. Cardiac Differentiation From iPSCs

FD-iPSCs and control-iPSCs were differentiated into cardiomyocytes (CMs) according to a previously established protocol (Lian et al., Directed cardiomyocyte differentiation from human pluripotent stem cells by modulating wnt/beta-catenin signaling under fully defined conditions. Nat Protoc. 2013; 8:162-175). The iPSCs were cultured on Geltrex-coated plates in mTeSR1 medium (STEMCELL Technology), and Accutase (Innovative Cell Technology) was used to detach the iPSCs from the plates. Then, the iPSCs were resuspended in mTeSR1 with 5 μM of Y27632 (Tocris Bioscience), a ROCK inhibitor, and were plated on Geltrex-coated plates. The culture medium was initially mTeSR1 and RPMI

(Life Technologies), with B-27 or without insulin (Life Technologies) with CHIR99021 (Selleckchem), a GASK3 inhibitor. After 24 hours, the medium was replaced with RPMI/B-27 without insulin. On day 3 of differentiation, combined medium was prepared by mixing the old medium with fresh RPMI/B-27 without insulin at a 1:1 ratio. The medium was replaced with combined medium containing 5 μM of IWP2 (Tocris Bioscience), a Wnt signaling inhibitor. On day 5 of differentiation, the medium was replaced with fresh RPMI/B-27 without insulin. RPMI with B-27 (Life Technologies) was added on day 7 of differentiation and changed every three days thereafter for three weeks.

### 6. Western Blot Assay

The extraction of proteins from cells and western blot analysis were performed as described previously (Yang et al., Inhibition of cancer stem cell-like properties and reduced chemoradioresistance of glioblastoma using microrna145 with cationic polyurethane-short branch pei. Biomaterials. 2012; 33:1462-1476). Whole cell lysates were separated by electrophoresis on 12% SDS-PAGE and transferred to a polyvinylidene fluoride membrane. The membranes were blocked with 5% nonfat milk at room temperature for 1 hour. The blots were incubated with primary antibodies in TBST buffer containing 3% nonfat milk at 4° C. overnight and subsequently with secondary antibodies conjugated with peroxidase at 25° C. for 1 hour. The immunoblots were developed using an enhanced chemiluminescence system, and the luminescence was visualized on X-ray film. The antibodies for western blotting are shown in Table 3.

TABLE 3
List of proteins tested by antibodies

| | | | | | Incubation |
|---|---|---|---|---|---|
| Protein | Assay | Antibody | Origin | Dilution | period |
| GAPDH | WB | rpab | Ab9385, Abcam, Inc | 1:5000 | 60 min |
| OCT3/4 | IF | rpab | #2840, Cell Signaling, Inc | 1:400 | O.N.* |
| NANOG | IF | rpab | #3580, Cell Signaling, Inc | 1:800 | O.N. |
| TRA 1-60 | IF | mmab | #4746, Cell Signaling, Inc | 1:1000 | O.N. |
| TRA 1-81 | IF | mmab | #4745, Cell Signaling, Inc | 1:1000 | O.N. |
| SMA | IF | rpab | Ab52218, Abcam, Inc | 1:500 | O.N. |
| NF | IF | mmab | MAB 1615, Millipore, Inc | 1:200 | O.N. |
| AFP | IF | mmab | #3903, Cell Signaling, Inc | 1:100 | O.N |
| HNF3β | IF | rpab | Sc-20692, Santa Cruz, Inc | 1:200 | O.N. |
| NESTIN | IF | rpab | ABD69, Millipore, Inc | 1:500 | O.N. |
| α-ACTININ | IF | mmab | Ab9465, Abcam, Inc | 1:200 | O.N. |
| MYL2 | IF | rpab | Ab79935, Abcam, Inc | 1:200 | O.N. |
| MYL7 | IF | mmab | Ab68086, Abcam, Inc | 1:200 | O.N. |
| cTNT | IF | mmab | Ab10214, Abcam, Inc | 1:200 | O.N. |
| GLA | WB | rpab | GTX101178, GeneTex, Inc | 1:1000 | 60 min |
| Crystallin A3 | WB | rpab | GTX109207, GeneTex, Inc | 1:1000 | 60 min |
| Crystallin A4 | WB | rpab | GTX109526, GeneTex, Inc | 1:1000 | 60 min |
| ALOX12 | WB, | rpab | PA5-26020, Thermo, Inc | 1:1000, | 60 min, |
| | IF | | | 1:200 | O.N. |
| ALOX15 | WB, | mmab | Ab119774, Abcam, Inc | | 60 min, |
| | IF | | | | O.N. |
| α-TUBULIN | IF | rpab | #5568, Cell Signaling, Inc | 1:1000 | 60 min. |

*O.N.: overnight.

### 7. Immunofluorescence Staining and Measurement of CM Size

The living cells and spheres were fixed in 4% paraformaldehyde, permeabilized in 0.1% Triton X-100, and blocked in 5% normal goat serum in PBS. The cells were incubated with primary antibodies, and the antibodies and conditions were shown in Table 3. After being washed three times with PBS, the cells were incubated with goat anti-mouse secondary antibodies conjugated with FITC (green) or PE (red). DAPI (blue) was used as the nuclear stain. Labeled cells were imaged with a laser-scanning confocal microscope (Olympus). The total amount of retained autofluorescent material was determined in the red (546) and green (488) channels by quantifying the pixel area (Adobe Photoshop/Image J software). The cellular area contents of the normal iPSC-CMs and the FD-iPSC-CMs were quantified using the ImageJ software package (National Institutes of Health, Bethesda, MD).

### 8. Transmission Electron Microscopy (TEM)

Cells (1×10⁷) were suspended in 1.2% agarose and fixed in 0.1 M phosphate buffer (PB), pH 7.4, containing 4% paraformaldehyde and 2.5% glutaraldehyde at 4° C. overnight. The samples were washed with 0.1 M PB before post-fixation with 1% OsO₄ in 0.05 M PB for 1 hour. After washing with distilled water, the samples were rinsed in block-stain with 0.2% uranyl acetate at 4° C. overnight. The samples were dehydrated in a serial dilution of ethanol for 10 min each (from 50% to 100% ethanol) and further infiltrated with a 100% ethanol/acetone (1:1) mixture and 100% acetone for 15 min each. Then, they were infiltrated with 100% acetone/Spurr resin (1:1) and (1:3) mixture for 1 hour each. The samples were changed to Spurr resin for continuous infiltration for 24 hours before being transferred to a capsule filled with Spurr resin. The Spurr resin was polymerized and solidified at 72° C. for 48 hours. The resin blocks were trimmed and cut using an ultramicrotome (Leica Ultracut R, Vienna, Austria). Thin sections were transferred to 200 mesh copper grids and stained with 2% uranyl acetate for 20 min and 2.66% lead citrate for 5 min prior to observation with a JEM1400 electron microscope (JEOL USA, Inc., Massachusetts, USA) at 100-120 kV.

### 9. Immunohistochemistry

Tissue specimens of patients with FD were collected and retrieved from the archives of the Department of Pathology of Taipei Veterans General Hospital, Taipei, Taiwan. The National Health Insurance (NHI) program in Taiwan, launched in 1995, has successfully provided comprehensive health care for all citizens. Dominant IVS4 mutation and cardiac involvement was predominantly found in Taiwan population. Since the enzyme replacement therapy (ERT) for FD with IVS4 mutation was only approved by Taiwan NHI using cardiac biopsy to confirm FD with cardiomyopathy, all FD cases enrolled in our study were confirmed by myocardium biopsy. The samples were collected using methods that conformed to the ethical guidelines of the Institutional Review Board. This study protocol was approved by the Institutional Review Board of Taipei Veterans General Hospital. A tissue array with heart tumor tissue and normal heart tissue (T301; Biomax, Inc., Rockville, Md.) was used as the control group. Immunohistochemistry was performed on 4-μm-thick paraffin-embedded sections of rectal specimens. After deparaffinization and dehydration, the specimens were boiled in 10 mM sodium citrate buffer (pH 6.0) for 40 minutes for antigen retrieval and then blocked in peroxidase-blocking solution (Dako Cytomation, Glostrup, Denmark). The primary antibody (listed in Table 3) was incubated at 4° C. overnight, and staining was detected using an Envision detection system (Envision detection system, peroxidase/DAB⁺, rabbit/mouse, Dako Cytomation). The specimens were counterstained with Mayer's hematoxylin.

### 10. Electrophysiological Examination

The recording area of probes with 64 recording electrodes (MED probe; MED-P515A, Alpha Med Scientific, Osaka) for the MED64 System (Alpha Med Scientific) was coated with 2 ml of 0.1% gelatin that had been incubated at 37° C. for 1 hour. Beating iPSC-derived CMs were transferred onto the MED-probe dishes, and the electrical potentials were recorded with the MED64 multi-electrode array system. The CMs spontaneously beat and showed CM-like action potential (average interval: 400 msec, overshoot: 20 mV). To evaluate the compounds, a cluster was treated with a compound in stepwise concentration increments. The medium was perfused at 1.7 ml/min at 37° C., and the field potentials were measured for 5 min. Then, medications, including isoproterenol (Proternol-LH, Kowa Pharmaceutical Company, Tokyo, Japan) and verapamil (Sigma-Aldrich, Mo., USA) were added to the medium (discrete colony samples were used for each drug) to measure the FPs for approximately 10 min.

### 11. Measurement of α-Gal A Enzyme Activity

Cells were washed twice with PBS, incubated in 200 μl of fresh medium at 37° C. and 5% CO₂ for 2 hours, and washed twice with PBS. Afterward, the cells were lysed in 60 μl of Lysis Buffer (27 mM of sodium citrate, 46 mM of sodium phosphate dibasic, 0.5% Triton X-100, pH 4.6). Lysates (10 μl) were added to 50 μl of Assay Buffer (Lysis Buffer without Triton X-100) containing 6 mM of 4-MUG and 117 mM of N-acetyl-D-galactosamine (GalNac, an inhibitor of -N-acetylgalactosaminidase, which is a lysosomal enzyme present in cell lysates that hydrolyzes 4-MUG) and incubated at 37° C. for 1 hour. The Stop Solution (0.4 M glycine, pH 10.8; 70 μl) was added, and the fluorescence was read on a Victor plate reader (Perkin-Elmer, Waltham, Mass.) at 355 nm excitation and 460 nm emission. A Micro BCA Protein Assay Kit (Pierce, Rockford, Ill.) was used according to the manufacturer's instructions to determine the protein concentration from 40 μl of cell lysate from each well. The total protein amount in each well was used to normalize the enzyme activity. A 4-methylumbelliferone (4-MU) standard curve ranging from 1.3 nM to 30 μM was run in parallel to calculate the absolute -Gal A activity, expressed as nmol of 4-MU released/mg protein/hour (nmol/mg protein/hour), which was normalized to the percentage of the untreated wild-type (% WT) enzyme activity.

### 12. Microarray Analysis and Bioinformatics

Total RNA was isolated using a standard Trizol protocol (Life Technologies, Bethesda, Md.) and the Qiagen RNAeasy (Qiagen, Valencia, Calif., USA) column for purification. RNA purity and quality were measured by a UV spectrophotometer and an Agilent 2100 Bioanalyzer (Agilent Technologies, Foster City, Calif.), and the RNA integrity number value was required to be >8 for each RNA sample. Ten to fifteen micrograms of total RNA from each sample was used in each cycle of microarray analysis. Affymetrix HG U133 Plus 2.0 microarrays containing 54,675 probe sets for >47,000 transcripts and variants, including 38,500 human genes, were used. A typical probe set contains 25-mer oligonucleotide pairs (a perfect match and a mismatch control). Some genes are measured by multiple probe sets. For microarray analysis, sample labeling, hybridization, and staining were carried out by Affymetrix standard protocol. Affymetrix.cel files were uploaded to R projector (R version 3.0.2; www.r-projector.org) and subsequently background corrected, normalized, and polished using robust multiarray averaging (RMA). Unsupervised principal component analysis was performed using all probe sets with a correlation matrix following z-normalization. A series of statistical filters, including fold changes and t-tests, were used to identify altered gene expression. To estimate the number of genes found by chance, we performed significance analyses of microarrays using RMA data with a false discovery rate. The default RMA settings were used to background correct, normalize and summarize all expression values. Significant difference between sample groups was identified using the ‘limma’ package of the Bioconductor. Briefly, a t-statistic was calculated as normal for each gene and a p-value then calculated using a modified permutation test. To control the multiple testing errors, a false discovery rate (FDR) algorithm was then applied to these p-values to calculate a set of q-values:

thresholds of the expected proportion of false positives, or false rejections of the null hypothesis. Heatmap was created by the dChip software (http://biosunl.harvard.edu/complab/dchip/). Principle component analysis (PCA) was performed also by the dChip software to provide a visual impression of how the various sample groups are related. Gene annotation and gene Ontology were performed by the DAVID Bioinformatics Resources 6.7 interface (http://david.abcc.ncifcrf.gov/). For obtaining functional regulatory networks, filtrated features from array analysis will be subjected into the plug-in of Cytoscape software (http://www.cytoscape.org/). The knowledge base behind Cytoscape was built upon scientific evidence, manually collected from thousands of journal articles, textbooks, and other data sources. After a list of signature genes was uploaded, interaction among focus genes and interaction among interacting genes and molecules from the knowledge base are used to combine genes into networks according to their probability of having more focus genes than expected by chance. The term “network” in Cytoscape is not the same as a biological or canonical pathway with a distinct function but a reflection of all interactions of a given protein as defined in the literature.

13. 1D Gel Electrophoresis (SDS-PAGE) and In-Gel dDgestion

Extracted proteins from FD-iPSC-CMs and control iPSC-CMs were denatured by boiling at 95° C. for 10 min. 1D gel electrophoresis was performed as described previously (Lai et al., Sry (sex determining region y)-box2 (sox2)/poly adp-ribose polymerase 1 (parp1) complexes regulate pluripotency. Proc Natl Acad Sci USA. 2012; 109:3772-3777) with 10% SDS-PAGE gels. After the gel was stained using a VisPRO 5-min protein staining kit (VP01-500; Visual Protein), each lane was cut into 10 equal sections, followed by reduction with J3-mercaptoethanol (1% vol/vol) in 25 mM ammonium bicarbonate at room temperature in the dark for 20 min and alkylation with 5% vol/vol 4-vinylpyridine in 25 mM ammonium bicarbonate for 20 min. Digestion was performed with 0.1% vol/vol proteomics grade modified trypsin (Sigma-Aldrich) in 25 mM of ammonium bicarbonate at 37° C. overnight. The extracts of trypsin-digested peptides were dried in a SpeedVac concentrator (Jouan, RC1022; Thermo Fisher Scientific).

### 14. LC-MS/MS Analysis

LC-MS/MS analysis was performed using an LTQ Orbitrap (Thermo Fisher Scientific). Each sample of digested peptides was reconstituted in 20 μl of 0.1% formic acid. The peptides were first injected into, and separated by, a nanoflow HPLC (Agilent 1100; Agilent Technologies) with a C18 column (75 μm ID×360 μm OD×15 cm; Agilent Technologies). The proteins became ionized particles after passing through the subsequent nanospray tip (New Objective). When operating the HPLC, the flow rate was set at 0.4 μl/min after a splitter. The LC gradient for the LC-MS/MS system ramped from 2-40% ACN in 120 min, and the system was used under the following conditions: automated data-dependent acquisition and 200-2000 m/z full scan mode for the three most intense peaks from each Orbitrap MS scan. Peptides with a +2 or +3 charge state were further subjected to CID. The spectra were obtained in raw data files with Xcalibur (version 2.0 SR2). Protein identification was accomplished via TurboSEQUEST (Thermo Fisher Scientific) using the UniProt database. A protein was confirmed once three peptides with Xcorr >2.5 were matched by sequencing.

### 15. ELISA-Based 15(S) Hydroxyeicosatetraenoic Acid (HETE) and 12(S)HETE Measurements

Whole blood samples were collected into 1.8 mg/ml EDTA-K3 tubes and centrifuged at 2500×g for 20 min at room temperature to obtain the plasma samples. The aliquots were stored at −80° C. until use. Enzyme-linked immunoassays (ELISAs) were used for 15(S) HETE (Cayman Chemicals) and 12(S) HETE (Detroit R&D) quantification according to the manufacturers' instructions (Detroit R&D).

### 16. Statistical Analyses

Statistical analysis was performed using SPSS software, version 13.0 (SPSS, Inc., Chicago, Ill., USA). The results are reported as the mean±SD. Variables with a normal distribution were compared with Student's t-test (two groups) or an ANOVA with post hoc LSD test (three groups). The comparison of the categorical variables was performed by Pearson's chi-squared (χ2) test. The results were considered statistically significant at P<0.05.

### II. Results

1. Clinical Findings and Generation of Fabry-iPSCs with GLA IVS4+919G>A Mutation

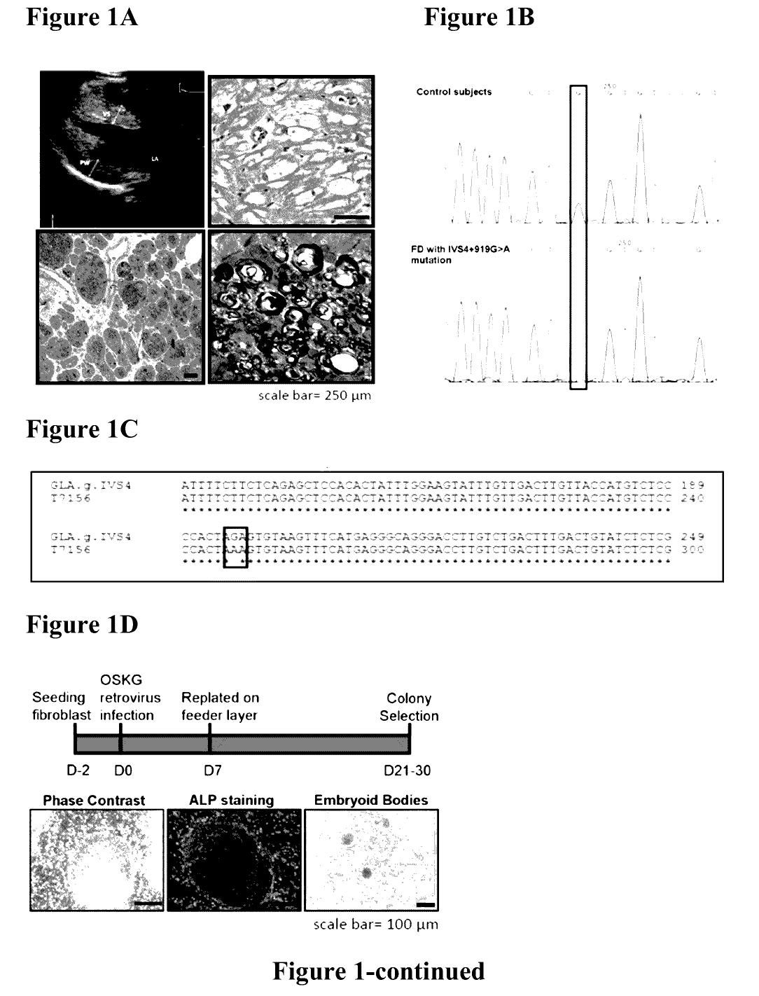

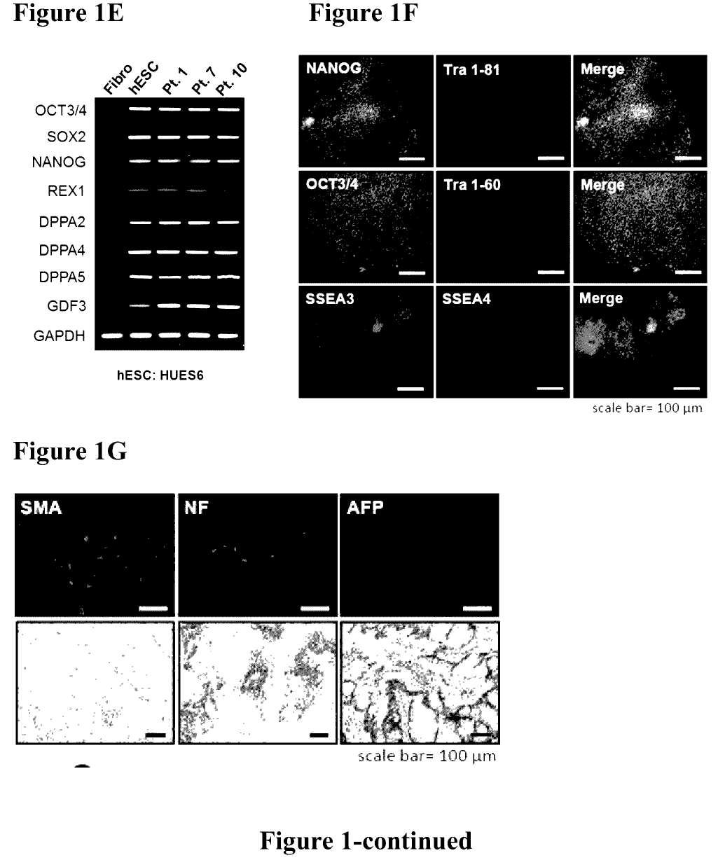

To explore the pathogenic relationship between late-onset GLA IVS4+919G>A mutation and cardiac FD, 10 FD patients (clinically presenting marked left ventricle (LV) hypertrophy, decreased GLA enzyme activity and genetically confirmed with the GLA IVS4+919G>A mutation were enrolled in this study. As shown in FIG. 1A and Table 4, which present the clinical findings of these ten FD patients, the transthoracic echocardiography demonstrated marked ventricular septum (VS) and posterior wall (PW) hypertrophy in left ventricle (LV). The histological findings showed sarcoplasmic vacuolization of the myocardial cells; the perinuclear vacuoles were filled with materials that stained positive for PAS and Sudan black. TEM analysis showed the presence of central vacuolar degeneration of myocytes with concentric lamellar structures (FIG. 1A, lower right). Furthermore, sequence analysis revealed the existence of a specific IVS4+919G>A mutation in the peripheral blood samples from FD patients (FIG. 1B and 1C). The patient information regarding the 10 FD patients validated who were validated for cardiomyopathy, lysosomal abnormalities and IVS4+919G>A mutation is also shown in Table 4.

TABLE 4
Summary of Information of Fabry disease patients with IVS4 + 919G > A mutation and
Characterization for FD-iPSC-derived cardiomyocytes

| | Patient | Patient | Patient | Patient | Patient | Patient | Patient | Patient | Patient | Patient |
|---|---|---|---|---|---|---|---|---|---|---|
| | 1 | 2 | 3 | 4 | 5 | 6 | 7 | 8 | 9 | 10 |
| Patient Information | | | | | | | | | | |
| Sex (F/M)¹/age | F/60 | M/67 | M/48 | M/65 | M/45 | M/49 | M/65 | M/74 | F/40 | F/61 |
| (year-old) | | | | | | | | | | |
| Marked ventricular | + | + | + | + | + | + | + | + | + | + |
| septum & posterior | | | | | | | | | | |
| wall hypertrophy in | | | | | | | | | | |
| LV | | | | | | | | | | |
| Positive stained for | + | + | + | + | + | + | + | + | + | + |
| PAS, Toluidine blue, | | | | | | | | | | |
| Sudan black | | | | | | | | | | |
| FD-Cardiomyopathy | + | + | + | + | + | + | + | + | + | + |
| by TEM | | | | | | | | | | |
| IVS4 + 919G > A | + | + | + | + | + | + | + | + | + | + |
| mutation | | | | | | | | | | |
| Establishment of | + | + | + | + | + | + | + | + | + | + |
| iPSC line | | | | | | | | | | |
| FD-iPSC-CM² | | | | | | | | | | |
| characterization | | | | | | | | | | |
| Cardiospecific | + | + | + | + | + | + | + | + | + | + |
| markers by IF³ | | | | | | | | | | |
| (MYL2, Actinin, | | | | | | | | | | |
| cTnT, MYL7) | | | | | | | | | | |
| IVS4 + 919G > A | + | + | + | + | + | + | + | + | + | + |
| mutation | | | | | | | | | | |
| Decreased GLA | + | + | + | + | + | + | + | + | + | + |
| mRNA and protein | | | | | | | | | | |
| Decreased α-GLA | + | + | + | + | + | + | + | + | + | + |
| activity | | | | | | | | | | |
| Cell enlargement & | + | + | + | + | + | + | + | + | + | + |
| hypertrophy | | | | | | | | | | |
| Lysosomal abnormal | Day | Day | Day | Day | Day | Day | Day | Day | Day | Day |
| accumulation of | 30 | 30 | 20 | 30 | 30 | 30 | 40 | 30 | 30 | 30 |
| glycosphingolipid by | | | | | | | | | | |
| TEM⁴ | | | | | | | | | | |

¹F: female, M: male
²FD-iPSC-CMs: FD-iPSC-derived cardiomyocytes.
³IF: immunofluorecence alaysis.
⁴Day 20: Post-induction 20 days; Day 30: Post-induction 30 days; Day 40: Post-induction 40 days.
TEM: transmission electric microscopy.
+positive finding.

To generate FD-iPSCs, skin fibroblasts from 10 FD patients with the IVS4+919G>A mutation were obtained for the iPSC generation (Table land Table 4). Because Glisl enhances the reprogramming efficiency of iPSCs along with the conventional factors Oct4/Sox2/Klf4, we used Oct4/Sox2/Klf4/Glisl to generate the FD-iPSCs. The skin-derived fibroblasts were transduced with a retroviral vector encoding Oct4/Sox2/Klf4/Glisl (Table 1). These cells were re-plated onto mitotically inactivated MEFs one week after transfection and were ready for the iPSC colony selection three week post-transfection (FIG. 1D). Subsequently, these colonies were cultivated on MEF feeder cells in KSR-based medium, generating various FD-iPSC clones that exhibited typical embryonic stem cell (ESC)-like morphology and positively stained for alkaline phosphate (ALP). To prevent MEF contamination, these FD-iPSCs were transferred to feeder-free culture in CSTI-8 medium without KSR supplementation. These feeder-free system-cultivated FD-iPSCs showed strong ALP positivity, and Ctrl-iPSCs yielded identical results (FIG. 1D, left and middle), indicating these cells were successfully reprogrammed to stem cells. RT-PCR indicated that the FD-iPSCs expressed several human ESC marker genes, including Oct3/4, Sox2, Nanog, Rex1, DPPA2, DPPA4, DPPAS and GDF3 (FIG. 1E). Immunofluorescence revealed that the FD-iPSCs strongly expressed Oct3/4, Nanog, SSEA3, SSEA4, Tra-1-60, and Tra-1-81 (FIG. 1F), confirming the stemness signature of these iPSCs. The FD-iPSCs were capable of embryoid body formation (FIG. 1D, right) and differentiation into smooth muscle cells, neuron-like cells, and hepatocyte-like cells (positive for SMA, NF, and AFP, respectively; FIG. 1G, upper). After implantation into immunocompromised mice, the FD-iPSCs formed teratomas containing the structures of tridermal lineages (FIG. 1G, lower), confirming these iPSCs could differentiate lineages for all three primary germ layers with pluripotent property. Chromosomal analysis indicated that the FD-iPSCs exhibited a karyotype identical to that of the control iPSCs reprogrammed from normal dermal fibroblasts (FIG. 1H). Additionally, we confirmed that all FD-iPSC lines carried the expected FD mutation (IVS4+919G>A) by genomic DNA sequencing analysis (FIG. 1I). Collectively, we established FD patient-specific iPSCs with the IVS4+919G>A mutation; these cells carry the original genetic mutation of FD, providing a further approach for investigating the pathogenic mechanism of FD.

2. Recapitulation of Cardiac GLA Abnormalities in FD-iPSC-CMs with IVS4+919G>A Mutation

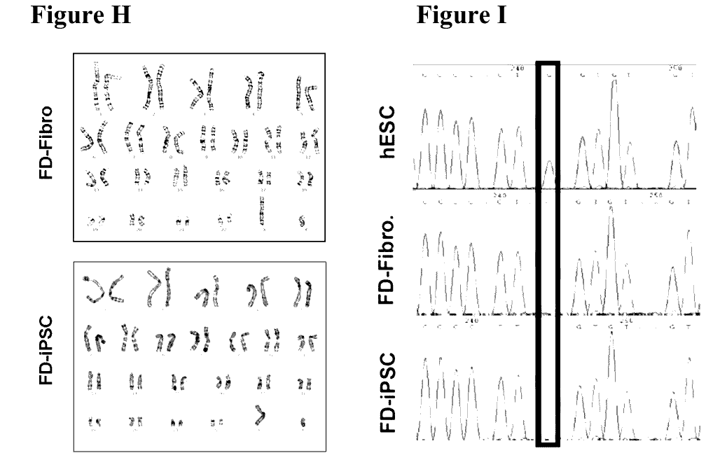

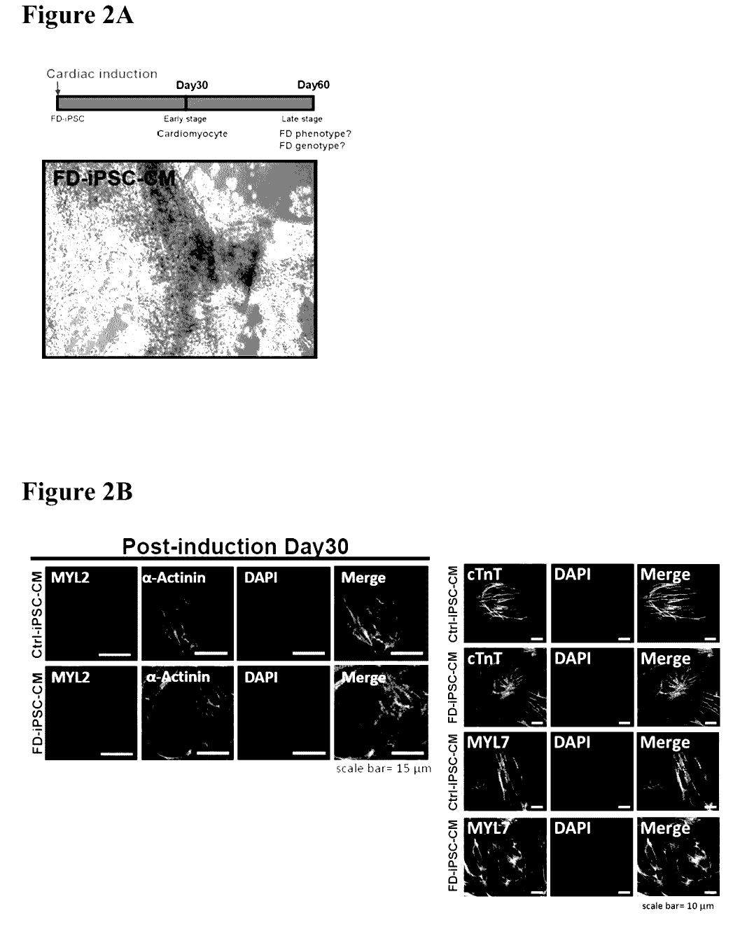

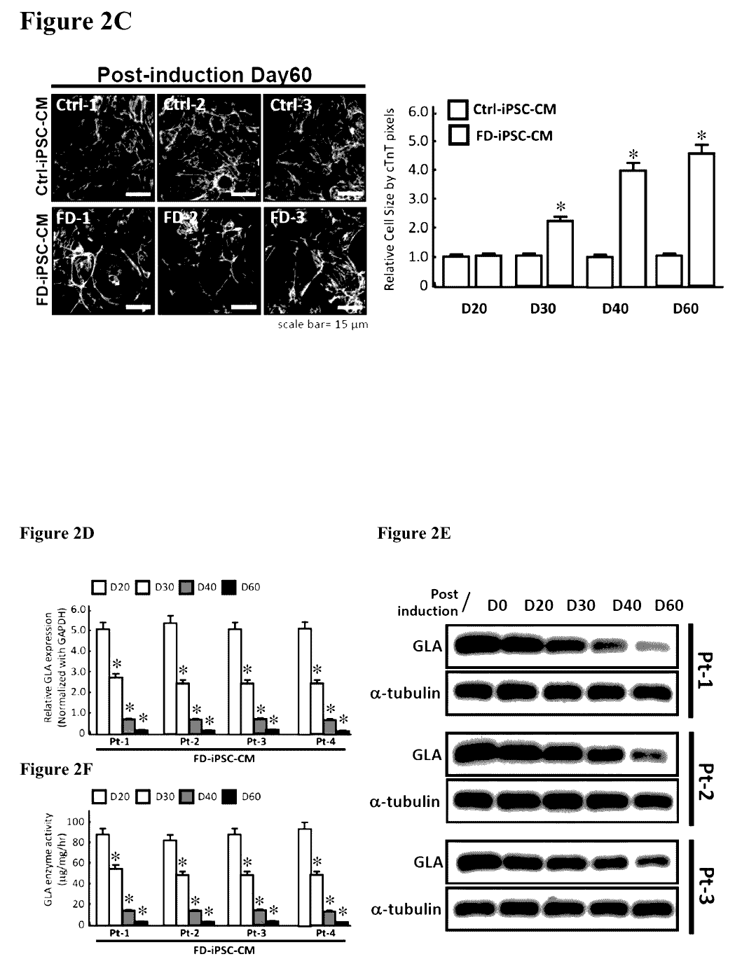

Patient-specific iPSC-CMs can recapitulate heart disease-specific features. However, whether these FD-iPSC-CMs could recapitulate FD-specific cardiomyopathy characteristics, especially the late-onset GLA IVS4+919G>A mutation in response to hypertrophic cardiomyopathy in middle-aged or elderly patients, remained unknown. We examined whether the FD-iPSC-CMs could express the pathophysiological features of FD at 20, 30, 40, and 60 days post-induction (early and late stage; FIG. 2A, upper). At 30 days post-induction, the FD-iPSC-CMs exhibited CM-like phenotypes (FIG. 2A, lower), synchronized beating (data not shown) and expression of cardio-specific markers including α-Actinin, MYL2, cTnT and MYL7 similar to the expression patterns of the Ctrl-iPSC-CMs (FIG. 2B). Because cellular hypertrophy is one of major features of FD-associated cardiomyopathy, we examined whether FD-iPSC-CMs recapitulated this important characteristics of Fabry disease. Quantification of the cTnT-positive margins revealed that the FD-iPSC-CMs were much larger than the Ctrl-iPSC-CMs at 60 days post-induction (FIG. 2C). Additionally, the FD-iPSCs expressed lower levels of GLA mRNA, GLA protein, and α-GLA enzyme activity than the Ctrl-iPSCs. To our interest, cardiac-specific induction further decreased α-GLA enzyme activity and levels of GLA mRNA in the FD-iPSCs (FIG. 2D, 2E, 2F). The maximal decrease in the GLA mRNA/protein and GLA activity was observed at 60 days post-induction (FIG. 2D, 2E, 2F). Together all, these data suggest that this iPSC-based platform reflects the typical characteristics of cellular hypertrophy and the deficient lysosomal GLA activity of FD.

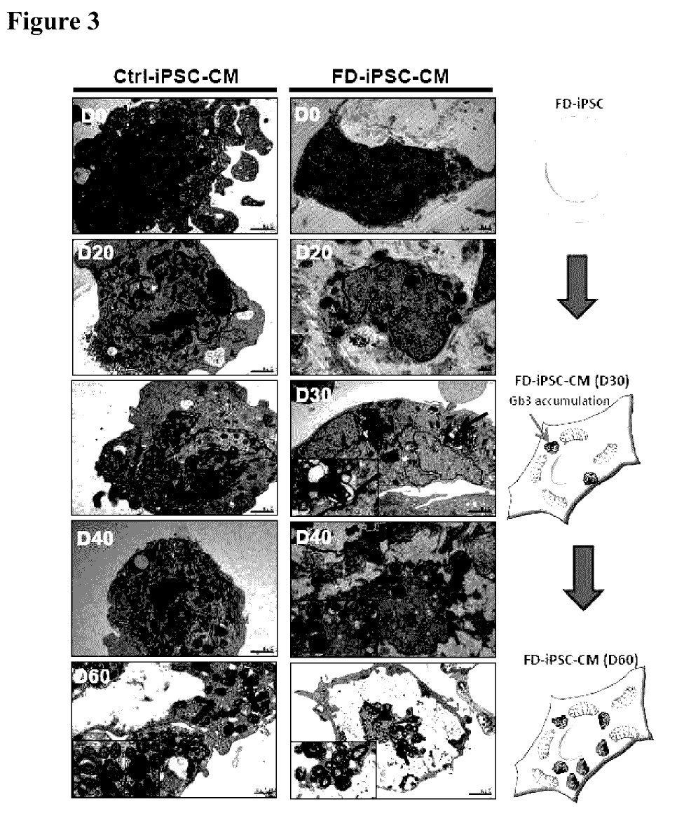

The deficiency of GLA results in the progressive accumulation of Gb3 with a characteristic zebra or onionskin appearance (a distinct limiting membrane and laminal structure with irregular alterations of dark and light zone) within different body cells. These Gb3 deposits are easily identified by electron microscopy. To demonstrate that FD-iPSC-CMs retain the typical pathological features of FD, we performed TEM to inspect the Gb3 accumulation in Ctrl-iPSC-CMs and FD-iPSC-CMs at 0, 20, 30, 40 and 60 days post-induction (FIG. 3). As shown in FIG. 3, no significant abnormalities or Gb3 accumulation was observed in either Ctrl-iPSCs-CMs or FD-iPSCs-CMs at post-induction days 0 and 20. After 30 days post-induction, several lysosomes that consisted of materials surrounded by a single-layer membrane were observed in the cytosol of FD-iPSC-CMs but not in the Ctrl-iPSC-CMs. Notably, at 60 days post-induction, the Gb3 deposits were more striking. These findings indicated that the pathophysiological characteristics of FD cardiomyopathy could be recapitulated by the in vitro platform of FD-iPSC-CMs. These late-stage FD-iPSC-CMs from all 10 patients displayed similar cardiac hypertrophy and TEM patterns (FIG. 3; Table 4), suggesting that our results show a high penetration and consistency with the clinical features of the FD-associated late-onset GLA IVS4+919G>A mutation.

3. FD-iPSC-CMs with IVS4+919G>A Mutation Exhibit Upregulated Cardiac Hypertrophy-associated Genes and Impaired Electrophysiological Response and Contractile Arrhythmia

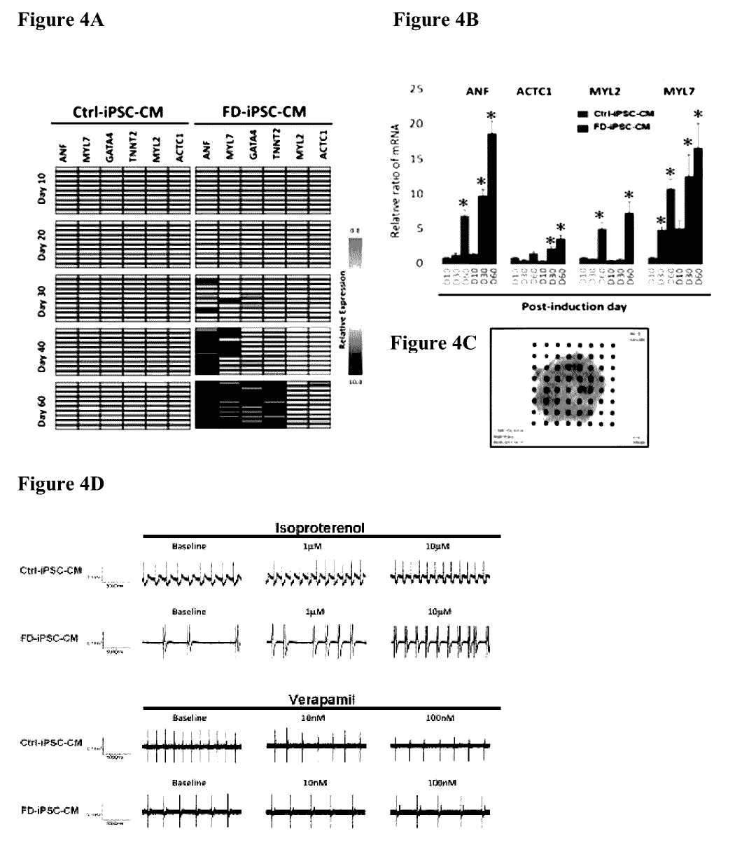

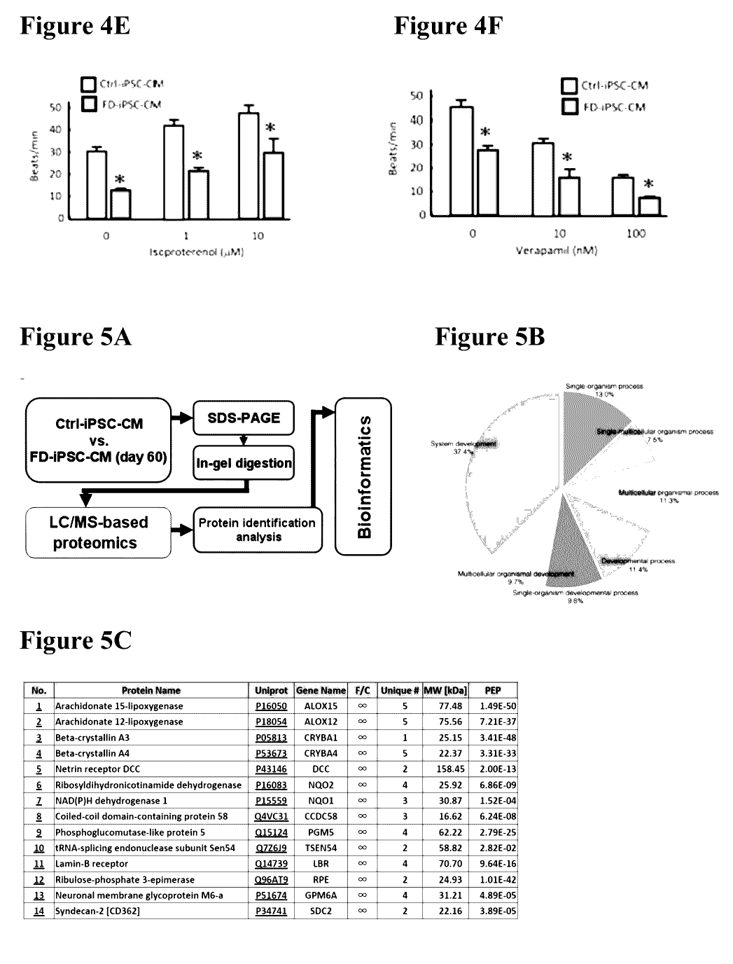

Considering the observation of enlargement of cell size in FD-iPSC-CMs than Ctrl-iPSC-CMs, we assessed if FD-iPSC-CM also express genomic pattern associated with cardiac hypertrophy. The genomic traits of Ctrl-iPSC-CMs and FD-iPSC-CM were examined using gene expression microarray analysis. The profiles of the differentially-expressed genes based on their functions in the Gene Ontology database were displayed in FIG. 4A. Remarkably, several cardiac hypertrophy-associated genes within the subset of FD-iPSC-CM were higher than that detected in Ctrl-iPSC-CMs at post-induction 30 days, and this upregulation were even higher at post-induction 40 and 60 days (FIG. 4A). Quantitative RT-PCR further indicated that several genes associated with cardiac hypertrophy, including ANF, ACTC1, MYL2, and MYL7, were largely upregulated at 30 days post-induction and reached maximal expression at 60 days post-induction (FIG. 4B). We further compared the electrophysiology of the Ctrl-iPSC-CMs and FD-iPSC-CMs using microelectrode array analysis with the MED system (Alpha MED Sciences; FIG. 4C). The differential responses to medications including the n-agonist (isoproterenol) and the Ca²⁺channel antagonist (verapamil), were measured in the Ctrl-iPSC-CMs and FD-iPSC-CMs (FIG. 4D). Isoproterenol led to an expected, dose-dependent chronotropic effect on the contractile colonies of Ctrl-iPSC-CMs, whereas FD-iPSC-CMs exhibited a basally lower beating frequency than Ctrl-iPSC-CMs and had random occurrences of arrhythmias (FIG. 4E). Furthermore, the treatment of verapamil led to maximal suppression of the beating frequency in response to approximately 10 μM of verapamil in Ctrl-iPSC-CMs, whereas FD-iPSC-CMs were almost completely unresponsive to verapamil (FIG. 4F). These results indicate that the FD-iPSC-CMs displayed characteristics of FD cardiomyopathy, including upregulated hypertrophic transcription factors, impaired regulation of electrophysiology, and impaired drug responses.

### 4. Proteomic Identification of Upregulation of Alox12 and Alox15 in FD-iPSC-CMs

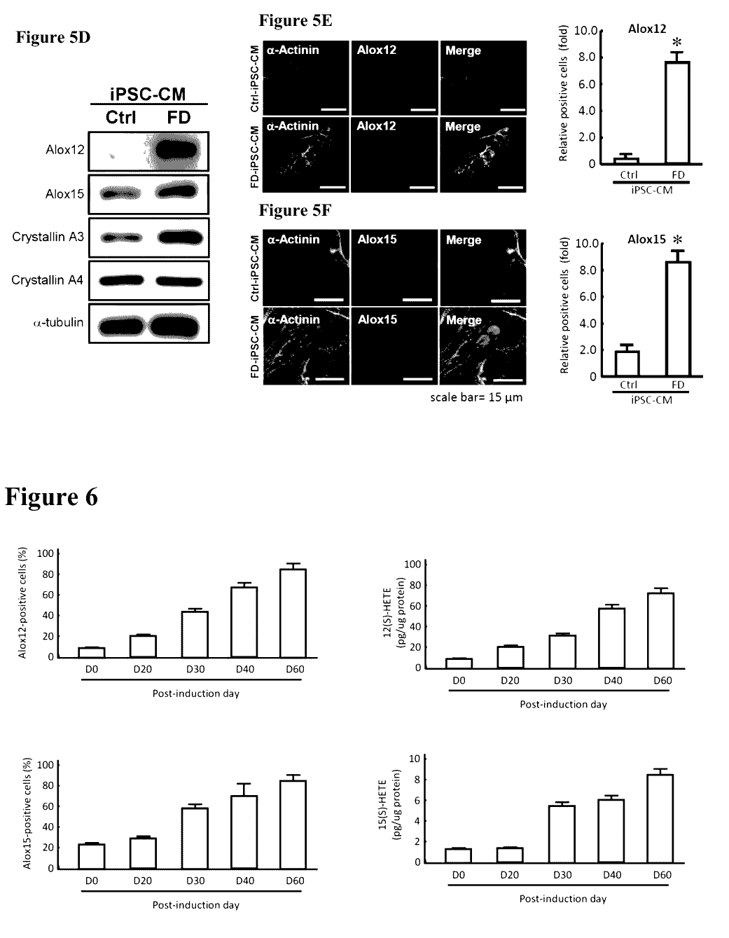

Proteomic analyses can provide high-throughput global screening for stem cell research. The novel reprogramming factors for iPSC generation was identified by MS-based proteomic analysis. Because our FD-iPSC-CM platform exhibited several phenotypes and gene-expression patterns compatible with the cardiac manifestations of FD patients, we performed a proteomic analysis to further investigate the potential mechanisms, and to screen for suitable markers of, FD with the IVS4+919G>A mutation. We first established the differential expression profiles of protein extracts from Ctrl-iPSC-CMs and FD-iPSC-CMs (60 days post-induction) using 1D liquid chromatography-tandem MS (LC-MS/MS, FIG. 5A). Based on the gene ontology (GO) database analysis, the predominant processes that were upregulated in the protein profiles of FD-iPSC-CMs included those pertaining to RNA processing, chromatin packaging and remodeling, cell structure and motility, and protein biosynthesis and those involved in mRNA transcription and DNA replication (FIG. 5B). Proteome screening found 20 significant candidate markers, including crystalline A3, crystalline A4, arachidonate 12-lipoxygenase (Alox12), and arachidonate 15-lipoxygenase (Alox15). Among these proteins, Alox12 and Alox15 are the most highly expressed proteins in FD-iPSC-CMs compared with Ctrl-iPSC-CMs (FIG. 5C). Western blotting also confirmed the upregulation of Alox12 and Alox15 in FD-iPSC-CMs (FIG. 5D). Notably, immunofluorescence further demonstrated the high expression of Alox12 and Alox15 in the intracellular compartment in FD-iPSC-CMs compared with normal iPSCs (FIG. 5E and 5F). Collectively, combining the patient-derived iPSC-CMs with proteomic identification provides a tool for exploring the pathophysiological mechanism for FD with the IVS4+919G>A mutation as well as a platform to search for novel biomarkers for FD cardiomyopathy.

5. High expression of Alox12/Alox15 and the Secretion of 12-HETE/15-HETE Induced by Gb3 in FD-iPSC-CMs Generated from Eight FD Patients

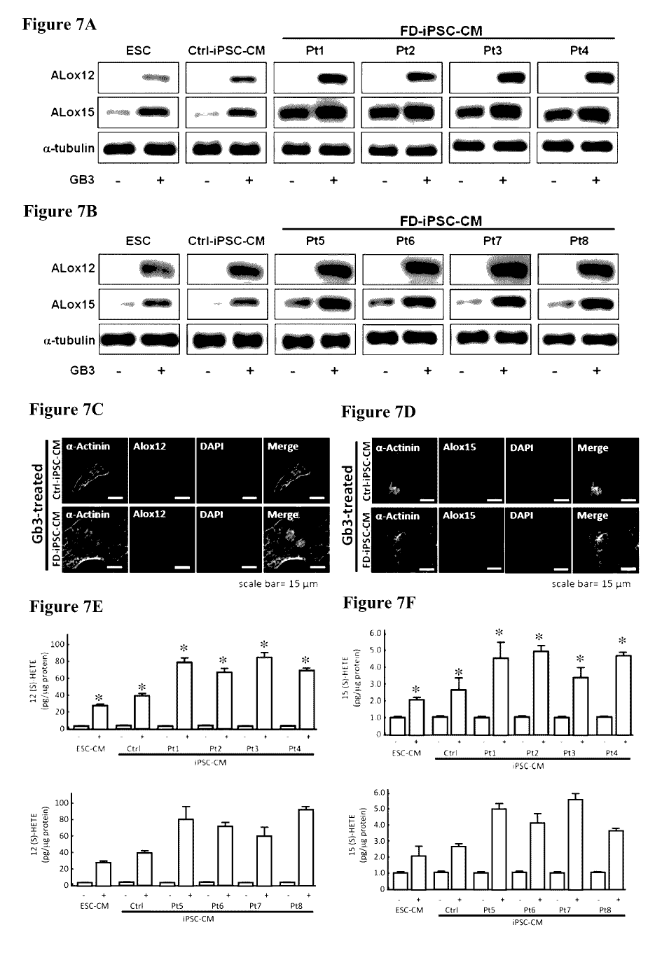

The lysosomal accumulation of glycosphingolipids, such as globotriaosylceramide (Gb3), is a potential inducer of FD-associated CM abnormalities. Recent studies have shown that Gb3 induces endothelial dysfunction through endosome-mediated lysosomal degradation (Choi et al., Globotriaosylceramide induces lysosomal degradation of endothelial kca3.1 in fabry disease. Arterioscler Thromb Vasc Biol. 2014; 34:81-89). However, whether Gb3 accumulation could be a pathogenic factor leading to Alox12/ALo15 upregulation in FD cardiomyopathy is unknown. We evaluated the protein expression pattern in response to the administration of Gb3 in FD-iPSC-CMs generated from eight FD patients with IVS4+919G>A mutation and defined cardiac abnormalities. First, as evaluated by immunofluorescence and ELISA, the employment of cardio-specific induction gradually increased the Alox12/Alox15 protein levels in FD-iPSC-CMs (Pt1 to Pt8) and promoted the secretion of 12-HETE and 15-HETE, the metabolites of Alox12 and Alox15, in a time-dependent manner, and the maximal expression levels of Alox12/15 were detected at late stage post-induction (days 30 to 60; FIG. 6). We next assigned FD-iPSC-CMs at post-induction day 20 (early stage) for the treatment of Gb3 and evaluated the Alox12/Alox15 expression patterns. Western blot demonstrated that Gb3 administration increased Alox12 and Alox15 in ESC-derived CMs (ESC-CMs), Ctrl-iPSC-CMs, and FD-iPSC-CMs. FD-iPSC-CMs (Pt1 to Pt8), while the same Gb3 treatment elicited the upregulation of Alox12/Alox15 at higher magnitudes than observed in ESC-CMs and Ctrl-iPSC-CMs (FIG. 7A, 7B). Immunofluoresence also indicated the same upregulation of Alox12/Alox15 in response to Gb3 treatment (FIG. 7C, 7D). 12-HETE and 15-HETE, play pivotal roles in cardiovascular diseases such as atherosclerosis and hypertension. To investigate the pathophysiological role and relationship between Alox12/Alox15 and 12-HETE/15-HETE in FD, we further used ELISA to assess the levels of secretion of 12-HETE/15-HETE in the conditioned medium of FD-iPSC-CMs. Gb3 mildly increase the secretion of 12-HETE/15-HETE from Ctrl-iPSC-CMs or ESC-CMs; however, the same treatment largely stimulated the secretion of 12-HETE and 15-HETE from FD-iPSC-CMs (Pt1 to Pt8) into the corresponding conditioned medium (FIG. 7E, 7F). This trend was consistent with the Gb3-induced upregulation of Alox12/15 in FD-iPSC-CMs (FIG. 7A, 7B). These findings revealed that Gb3 is a potent inducer of Alox12/Alox15 and the metabolites, 12-HETE/15-HETE in FD-iPSC-CMs. Because Gb3 accumulation serves critical roles in FD, these in vitro data support the hypothesis that Alox12/Alox15 expression and 12-HETE/15-HETE secretion are markers for FD-associated CM abnormalities.

### 6. Alox12/Alox15 as Potential Markers of FD-Associated Cardiomyopathy

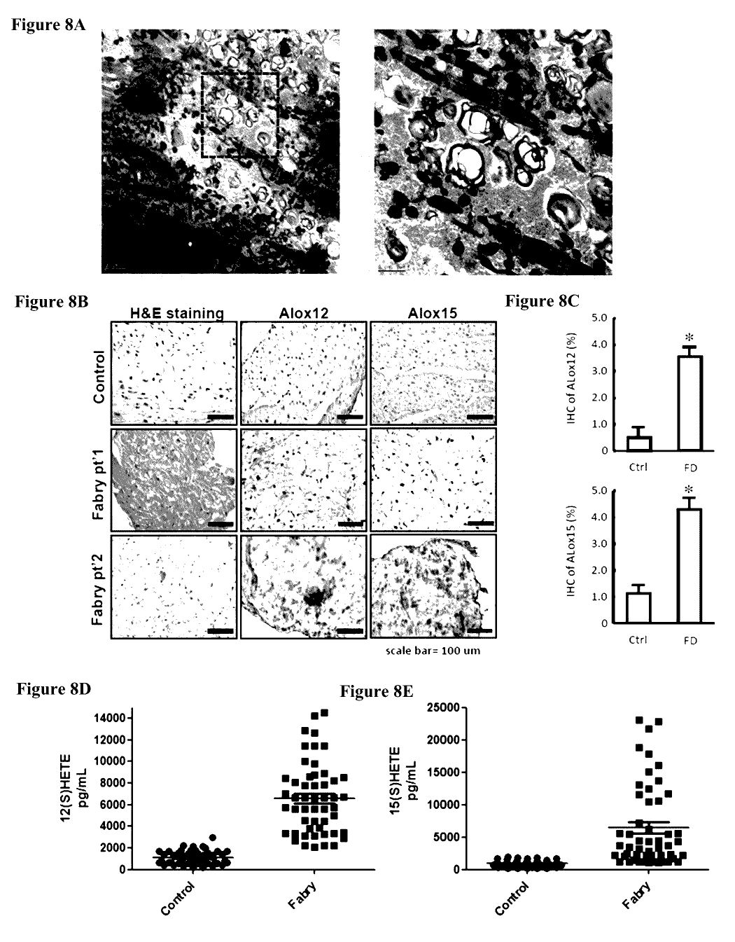

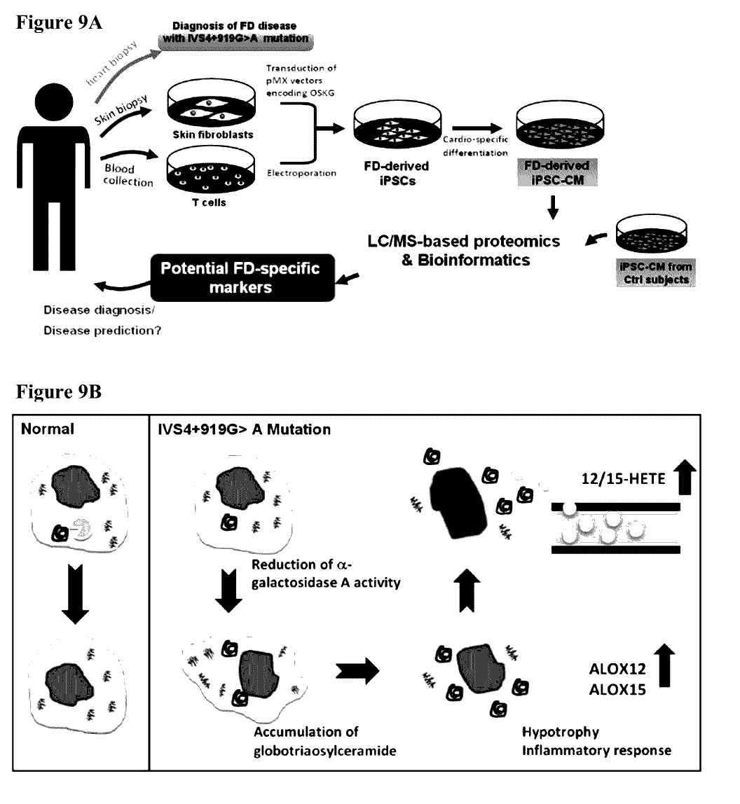

Based on our in vitro observation of the upregulation of Alox12/Alox15 and high secretion of their metabolites 12-HETE/15-HETE in FD-iPSCs-CMs, we next evaluated whether Alox12/Alox15 and the circulatory 12-HETE/15-HETE levels could be used as a potential diagnostic markers for FD-associated cardiac complications. Cardiac biopsy sample from five FD patients with IVS4+919G>A mutation, typical cardiac symptoms and Gb3 deposits in myocardium (FIG. 8A) were examined for the cardiac expression of Alox12/Alox15. A remarkable number of Alox12- and Alox-15 -positive cells were detected in the cardiac biopsy sample compared with the normal heart samples (Heart tumor test tissue array T301, provided by Us Biomax Inc., Rockville, Md.; FIG. 8B, 8C). Furthermore, to investigate the relationship between Alox12/15 expression and 12-HETE/15-HETE secretion in FD patients with IVS4+919G>A mutation, we measured the serum levels of 12-HETE/15-HETE in 51 FD patients with the IVS4+919G>A mutation who had significant LV hypertrophy (Table 5). The serum levels of 12-HETE and 15-HETE in these patients were significantly higher than in the normal age- and sex-matched subjects (n=51; FIG. 8D, 8E). These findings strongly indicated that Alox12/15 and their circulatory metabolites 12-HETE/15-HETE could be used as a potential diagnostic markers in FD-patients with the IVS4+919G>A mutation (FIG. 9).

TABLE 5
Patient information for 51 FD patients with IVS4 + 919 G > A
mutation and serum levels of HETE-12/15

| Patient information¹ | Fabry Disease (FD) | Control | P |
|---|---|---|---|
| Age, years | 52 ± 14³ | 52 ± 13 | NS⁴ |
| Patient numbers | 51 | 51 | NS |
| Gender | 31:20 | 27:24 | NS |
| (Female:Male) | | | |
| Parameters of | | | |
| LV hypertrophy² | | | |
| IVS, mm | 11.7 ± 1.0 | 8.7 ± 1.9 | <0.001 |
| LPW, mm | 11.8 ± 3.2 | 8.4 ± 1.5 | <0.001 |
| LVM, g | 188 ± 82 | 130 ± 42 | <0.001 |
| HETE-12, pg/mL | 7341 ± 908 | 1246 ± 345 | <0.001 |
| HETE-15, pg/mL | 6627 ± 894 | 2011 ± 561 | <0.001 |

¹Patient information of 51 FD patients (IVS4 + 919 G > A mutation) and 51 donors with age, gender-matched controls.
²LV: left ventricle; IVS: intraventricular septum; LPW: left posterior wall; LVM: left-ventricular mass.
³Data are mean ± SD.
⁴NS: indicates no significance.

### II

### I. Materials and Methods

1. Generation of FC Patient-Specific iPSCs

This study followed the tenets of the Declaration of Helsinki, and the protocols and procedures were approved by the board of the Taipei Veterans General Hospital. As for generation of patient-specific iPSCs, total of 12 Fabry disease patients with cardiomyopathy (FC) were enrolled in this study (Table 6). Isolated T cells from patients' peripheral blood (10 ml) were reprogrammed into iPSCs via integration-free episomal vectors. Using T cell-reprogrammed iPSCs, we generated FC-specific iPSC-CMs (FC-iPSC-CMs) that provided a high-throughput platform for exploring the potential pathogenesis of FC. FC-iPSCs and control-iPSCs were differentiated into cardiomyocytes (CMs) according to a previously established protocol. The FD-iPSCs or control-iPSCs were cultured on Geltrex-coated plates in mTeSR1 medium (STEMCELL Technology), and Accutase (Innovative Cell Technology) was used to detach the iPSCs from the plates. The iPSCs were then re-suspended in mTeSR1 with 5 μM of Y27632 (Tocris Bioscience), a ROCK inhibitor, and plated on Geltrex-coated plates. The culture medium was initially mTeSR1 and RPMI (Life Technologies), with B-27 or without insulin (Life Technologies) with CHIR99021 (Selleckchem), a GASK3 inhibitor. After 24 hours, the medium was replaced with RPMI/B-27 without insulin. On day 3 of differentiation, combined medium was prepared by mixing the old medium with fresh RPMI/B-27 without insulin at a 1:1 ratio. The medium was replaced with combined medium containing 5 μM of IWP2 (Tocris Bioscience), a Wnt signaling inhibitor. On day 5 of differentiation, the medium was replaced with fresh RPMI/B-27 without insulin. RPMI with B-27 (Life Technologies) was added on day 7 of differentiation and changed every three days thereafter for three weeks.

TABLE 6
The clinical information and Characterization and establishment of induced pluripotent stem cell
(iPSC) derived from 12 FD patients

| | No. 1 | No. 2 | No. 3 | No. 4 | No. 5 | No. 6 |
|---|---|---|---|---|---|---|
| Patient | | | | | | |
| Sex, age | Female, | Male, | Male, | Male, | Male, | Male, |
| | 60 | 67 | 48 | 65 | 45 | 49 |
| Marked ventricular septum & | + | + | + | + | + | + |
| posterior wall hypertrophy in LV | | | | | | |
| Positive stained for PAS, | + | + | + | + | + | + |
| Toluidine blue, Sudan black | | | | | | |
| FC-Cardiomyopathy by TEM | + | + | + | + | + | + |
| Establishment of iPSC line | + | + | + | + | + | + |
| Original of samples | PB | PB | PB | PB | PB | PB |
| Reprog-Efficiency (OSKG) | 0.2% | 0.3% | 0.05% | 0.4% | 0.2% | 0.6% |
| Cell passages of IPSC | 50 | 35 | 30 | 30 | 20 | 20 |
| FS-iPSC characterization | | | | | | |
| ALP staining | + | + | + | + | + | + |
| qPCR (Stemness genes) | + | + | + | + | + | + |
| Immunofluorescence | + | + | + | + | + | + |
| (O/N/S-3/S-4/T-81/T-60) | | | | | | |
| Karyotype | normal | normal | normal | normal | normal | normal |
| Tridermal Differentiation | + | + | + | + | + | + |
| In vivo teratoma formation | + | + | + | + | + | + |
| Mutation type | IVS4 + | Classic | IVS4 + | IVS4 + | Classic | Classic |
| | 919G > A | type | 919G > A | 919G > A | type | type |
| | No. 7 | No. 8 | No. 9 | No. 10 | No. 11 | No. 12 |
| Patient | | | | | | |
| Sex, age | Male, | Male, | Female, | Female, | Female, | Female, |
| | 65 | 74 | 40 | 61 | 48 | 64 |
| Marked ventricular septum & | + | + | + | + | + | + |
| posterior wall hypertrophy in LV | | | | | | |
| Positive stained for PAS, | + | + | + | + | + | + |
| Toluidine blue, Sudan black | | | | | | |
| FC-Cardiomyopathy by TEM | + | + | + | + | + | + |
| Establishment of iPSC line | + | + | + | + | + | + |
| Original of samples | PB | PB | PB | PB | PB | PB |
| Reprog-Efficiency (OSKG) | 0.3% | 0.5% | 0.5% | 0.1% | 0.4% | 0.2% |
| Cell passages of IPSC | 15 | 15 | 15 | 12 | 12 | 12 |
| FS-iPSC characterization | | | | | | |
| ALP staining | + | + | + | + | + | + |
| qPCR (Stemness genes) | + | + | + | + | + | + |
| Immunofluorescence | + | + | + | + | + | + |
| (O/N/S-3/S-4/T-81/T-60) | | | | | | |
| Karyotype | normal | normal | normal | normal | normal | normal |
| Tridermal Differentiation | + | + | + | + | + | + |
| In vivo teratoma formation | + | + | + | + | + | + |
| Mutation type | IVS4 + | IVS4 + | IVS4 + | IVS4 + | IVS4 + | IVS4 + |
| | 919G > A | 919G > A | 919G > A | 919G > A | 919G > A | 919G > A |

PB: Peripheral blood.
Reprog-Efficiency: Reprogramming efficiency.
Cell passages of iPSC: iPSCs remained stable through 30 passages and showed ESC-like pluripotent property.
O/N/S-3/S-4/T-81/T-60: Oct4, Nanog, SSEA3, SSEA4, Tra 1-81, Tra 1-60.
ALP: Alkaline phosphatase

2. Enrolled Study subjects and ERT Treatment

All procedures of tissues collection followed the tenets of the Declaration of Helsinki and were reviewed by Institutional Review Committee at Veterans General Hospital in Taiwan. A total of 47 Fabry-associated cardiomyopathy (FC) patients that have received ERT (intravenous injection of •—Gal) were enrolled in this study. Fabry-associated cardiomyopathy (FC) diagnosed in the 47 patients was confirmed in Taipei Veterans General Hospital by genetic mutation analysis, transthoracic echocardiography, decrease of GLA activity in serum samples, Gb3 accumulation in cardiac biopsy, and typical FC clinical characteristics under CMR-LGE scan (Table 7). The enzyme replacement therapy (ERT) administration in patients with FC was supported by Ministry of Health and Welfare in Taiwan based on Gb3 accumulation in cardiac biopsy, and executed by intravenously administration of Agalsidase alfa (Replagal® 0.2 mg/kg or agalsidase beta—Fabrazyme® 1.0 mg/kg) at a two-week interval. Echocardiography and CMR-LGE were used to validate the ERT efficacy and prognosis. The ERT treatment was supported by Ministry of Health and Welfare in Taiwan. Agalsidase alfa (Replagal® 0.2 mg/kg) and was administered intravenously in all of 47 FC patients at a two-week interval for 1 to 3.6 years (mean F/U time: 2.2±1.1 years; range: 1.0 - 3.6 years).

TABLE 7
The clinical characteristics, Cardiac MRI, and Serological Biomarker Results of Fabry-cardiomyopathy Patients

| | Fibrosis | Non-Fibrosis | |
|---|---|---|---|
| | (N = 17) | (N = 30) | P-value |
| Age, yrs | 61 ± 6.3 | 60 ± 5.8 | 0.09 |
| Women, no. (%) | 2 (11%) | 4 (13%) | 0.07 |
| Body mass index (BMI), g/m² | 24.7 ± 2.4 | 23.3 ± 3.2 | 0.07 |
| MSSI Cardiovascular score | 13.5 ± 1.7 | 13.2 ± 2.1 | 0.08 |
| Hypertension, no. (%) | 9 (52%) | 15 (50%) | 0.07 |
| Coronary artery disease, no. (%) | 3 (17%) | 6 (20%) | 0.08 |
| Left ventricular hypertrophy (LVH), no. (%) | 17 (100%) | 30 (100%) | 0.21 |
| Diabetes, mellitus no. (%) | 5 (29%) | 9 (30%) | 0.18 |
| Creatinine Clearance | | | |
| CCr: >60 ml/min, no. (%) | 5 (29%) | 5 (29%) | 0.19 |
| Ccr: 30-60 ml/min, no. (%) | 12 (70%) | 12 (70% ) | 0.22 |
| Left ventricular mass (LVH), g | 187.2 ± 12.8 | 182 ± 10.2 | 0.09 |
| Left ventricular mass index (LVMI) | 62 ± 7 | 65 ± 1 | 0.08 |
| Left ventricular ejection fraction (LVEF), % | 70% | 74% | 0.09 |
| Interventricular septum (IVS), mm | 15.5 ± 0.4 | 13.8 ± 0.5 | 0.06 |
| LysoGB3, nM | 3.5 ± 1.7 | 3.9 ± 1.5 | 0.06 |
| 12(S)-HETE, ng/mL | 3710 ± 40.2 | 2011 ± 21.6 | <0.01 |
| 15(S)-HETE, ng/mL | 333.8 ± 4.1 | 180.3 ± 2.8 | <0.01 |
| ERT follow-up time, years | 2.1 ± 0.8 | 2.3 ± 1.3 | 0.08 |
| | (1.3-2.9) | (1-3.6) | |

MSSI: The Mainz Severity Score Index.

### 3. Standard Echocardiographic Measurements and Cardiac MRI Study

LV end-diastolic and end-systolic dimensions and end-diastolic thickness of the posterior wall (PWT) and septum were measured with standard M-mode echocardiographic methods (Artida, Toshiba Medical Systems, Tokyo, Japan). Myocardial mass was calculated with the Devereux formula. Ejection fraction was calculated with the modified Simpson method. Early (E) and late (A) diastolic trans-mitral inflow velocity (MV A) and the ratio (E/A) were sampled by a pulse wave Doppler between the tips of the anterior and posterior mitral leaflets. Deceleration time (DT) of the early diastolic flow was also measured. Routine cine MRI with gadolinium was carried out in all patients with Fabry disease as part of the standard assessment. The LGE technique was applied to detect changes in tissue integrity in the LV myocardium.

### 4. Measurement of α-Gal A Enzyme Activity and Alox12/15

Lysates (100) were added to 50 μl of Assay Buffer (Lysis Buffer without Triton X-100) containing 6 mM of 4-MUG and 117mM of N-acetyl-D-galactosamine and incubated at 37° C. for 1 hour. The Stop Solution (0.4M glycine, pH 10.8; 700) was added, and the fluorescence was read on a Victor plate reader (Perkin-Elmer, Waltham, Mass.) at 355 nm excitation and 460nm emission. The enzyme activity was normalized by total amount of protein.

### 5. LysoGB3 Analysis

For liquid chromatography for lysoGb3 and Gb3 analysis, we used Waters Alliance 2795XE HPLC system to perform stepwise gradient elution. The flow rate was 0.3 mL/min in lysoGb3 or Gb3 experiment. Mass spectrometry of lysoGb3 and Gb3 detection was performed in positive ion mode (ES+) on a triple quadruplemass spectrometer (Quattro Ultima, Waters, Milford, Mass.) with NeoLynx software version 4.1. The analyzing methods were modified from the protocol provided by Shire Human Genetic Therapies. LC-MS/MS analysis was performed using an LTQ Orbitrap (Thermo Fisher Scientific) as described previously.

### 6. ELISA-Based 15(S)-HETE and 12(S)-HETE Measurements

Whole blood samples were collected into 1.8 mg/ml EDTA-K3 tubes and centrifuged at 2500×g for 20 min at room temperature to obtain the plasma samples. The aliquots were stored at −80° C. until use. Enzyme-linked immunoassays (ELISAs) were used for 15(S) HETE (Cayman Chemicals) and 12(S) HETE (Detroit R&D) quantification according to the manufacturers' instructions (Detroit R&D).

### 7. Statistical Analysis

For the human subject data, the variables are presented as the mean±standard deviation and compared with Student's t-test. A paired t-test was used to evaluate ERT efficacy. We used a linear regression model to explore the associations between the changes in the levels of the biomarkers and left ventricular mass index (LVMI) before and after ERT. The results were considered to be significant at P<0.05.

### II. Results

1. Recapitulation of Cardiac Abnormalities with GB3 Accumulation in FC Patient-Specific iPSC-Derived Cardiomyocytes (iPSC-CMs)

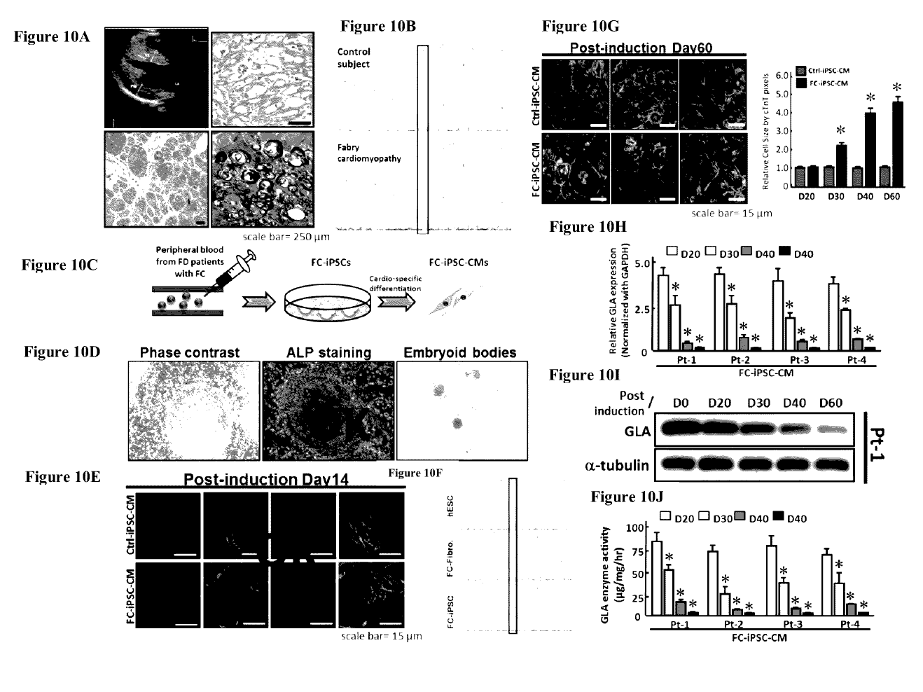

Cellular reprogramming technology and patient-specific iPSCs provide an opportunity to overcome the current limitations in investigating inherited lysosomal storage disorders. We collected 12 patients with late-onset Fabry cardiomyopathy (FC) who were diagnosed by transthoracic echocardiography and cardiac biopsy, and the specific gene mutation was validated by sequence analysis (FIG. 10A and 10B). FC-iPSCs were generated from patient-derived T cells using the electroporation transfection method (FIG. 10C; Table 6 and 8), and both Ctrl-iPSCs and FC-iPSCs exhibited iPSC characteristics including positive staining for ALP, embryonic stem-like morphologies (FIG. 10D), expression of stemness genes (Oct3/4, Nanog, and SSEA3; Online Table 6), regular reprogramming efficiency, karyotyping, and teratoma formation. We induced myocardial differentiation of FC-iPSCs and found that these differentiated cardiomyocytes (FC-iPSC-CMs) expressed cardiac-specific markers including α-actinin and MYL2 (FIG. 10E), exhibited CM-like phenotypes, and displayed a synchronized beating feature. Moreover, genomic DNA sequencing analysis confirmed that all FC-iPSC-CMs consistently carried the FD-specific mutation (FIG. 10F; Table 6 and 8).

TABLE 8
Summary of Information of FC patients and Characterization for
FC-iPSC-derived cardiomyocytes

| Sex, age (year-old), | Female, | Male, | Male, | Male, | Male, | Male, |
|---|---|---|---|---|---|---|
| FD | 60 | 48 | 67 | 65 | 45 | 49 |
| Mutation type | IVS4 + | Classic | IVS4 + | IVS4 + | Classic | Classic |
| | 919G > A | type | 919G > A | 919G > A | type | type |
| Establishment of | + | + | + | + | + | + |
| iPSC line | | | | | | |
| (Refer to Suppl | | | | | | |
| table 2) | | | | | | |
| FD-iPSC-CM | | | | | | |
| characterization | | | | | | |
| Cardiospecific | + | + | + | + | + | + |
| markers by IF | | | | | | |
| (MYL2, Actinin, | | | | | | |
| cTnT, MYL7.) | | | | | | |
| IVS4 + 919G > A | + | + | + | + | + | + |
| mutation | | | | | | |
| Decreased GLA | + | + | + | + | + | + |
| mRNA and protein | | | | | | |
| Decreased α-GLA | + | + | + | + | + | + |
| activity | | | | | | |
| Cell enlargement & | + | + | + | + | + | + |
| hypertrophy | | | | | | |
| Lysosomal abnormal | + | + | + | + | + | + |
| accumulation of | | | | | | |
| glycosphingolipid | | | | | | |
| by TEM | | | | | | |
| Sex, age (year-old), | Male, | Male, | Female, | Female, | Female, | Male, |
| FD | 65 | 74 | 40 | 61 | 66 | 51 |
| Mutation type | IVS4 + | IVS4 + | IVS4 + | IVS4 + | IVS4 + | IVS4 + |
| | 919G > A | 919G > A | 919G > A | 919G > A | 919G > A | 919G > A |
| Establishment of | + | + | + | + | + | + |
| iPSC line | | | | | | |
| (Refer to Suppl | | | | | | |
| table 2) | | | | | | |
| FD-iPSC-CM | | | | | | |
| characterization | | | | | | |
| Cardiospecific | + | + | + | + | + | + |
| markers by IF | | | | | | |
| (MYL2, Actinin, | | | | | | |
| cTnT, MYL7.) | | | | | | |
| IVS4 + 919G > A | + | + | + | + | + | + |
| mutation | | | | | | |
| Decreased GLA | + | + | + | + | + | + |
| mRNA and protein | | | | | | |
| Decreased α-GLA | + | + | + | + | + | + |
| activity | | | | | | |
| Cell enlargement & | + | + | + | + | + | + |
| hypertrophy | | | | | | |
| Lysosomal abnormal | + | + | + | + | + | + |
| accumulation of | | | | | | |
| glycosphingolipid | | | | | | |
| by TEM | | | | | | |

FC: Fabry cardiomyopathy.
FC-iPSC-CMs: FC-iPSC-derived cardiomyocytes.
IF: immunofluorecence analysis.
+positive finding.
TEM: transmission electric microscopy.

Patient-derived iPSCs chronologically exhibit typical characteristics of cardiomyopathy, such as familial hypertrophic cardiomyopathy (FHC), after a defined period of cardiac differentiation. FC is a late-onset cardiac manifestation with massive Gb3 accumulation and cardiomyocyte hypertrophy. We sought to investigate whether FC-iPSC-CMs could recapitulate the pathophysiological characteristics of Fabry-specific cardiomyopathy. A quantification of the cTnT-positive margins revealed that the cell size of the FC-iPSC-CMs was increased compared with Ctrl-iPSC-CMs 40 to 60 days after myocardial differentiation (FIG. 10G; Table 6), indicating the development of cardiomyocyte hypertrophy in FC-iPSC-CMs. As measured by quantitative RT-PCR and microarray analysis, the mRNA expression levels of several cardiac hypertrophy-associated genes (ANF, ACTC1, MYL2, and MYL7) were also increased in FC-iPSC-CMs compared with Ctrl-iPSC-CMs 30 days post-differentiation, and this upregulation continued up to 60 days post-differentiation. Moreover, during the course of differentiation, the mRNA and protein expression levels of GLA (FIG. 10H and 10I), as well as the enzyme activity of GLA (FIG. 10J), were all declined and reached the maximal decrease at day 60 post-induction.

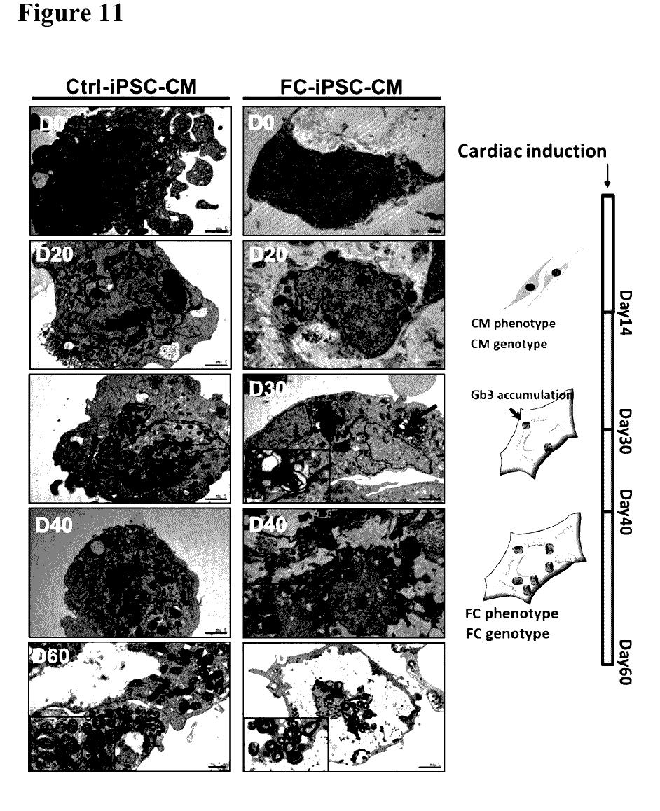

Gb3 accumulation in myocardial tissues is the prominent pathological phenotype for FC. To further examine whether FC-iPSC-CMs retain this typical pathological feature of FC, we performed a time-course transmission electron microscope (TEM) inspection of the Gb3 accumulation in Ctrl-iPSC-CMs and FD-iPSC-CMs. Gb3 accumulation started to be observed in FC-iPSCs-CMs 20 days post-differentiation but not in Ctrl-iPSC-CMs (FIG. 11). The Gb3 accumulated in differentiated FC-iPSCs-CMs became significant at day 30 and turned to be more prominent at 40 to 60 days post-differentiation (FIG. 11). The chronic intracellular accumulation of Gb3 at 40 to 60 days post-differentiation, as well as cardiomyocyte hypertrophy and FC-associated phenotypes (Tables 6 and 8), were repeatable in all the 12 patient-derived FD-iPSC-CMs including 9 with the IVS4+919G>A mutation and 3 with classic FD. These phenomena were highly compatible with the late-onset cardiac manifestation of Left ventricular hypertrophy (LVH) of Fbary-associated cardiomyiopathy (FIG. 11). Collectively, these data demonstrate the feasibility of FC-iPSC-CM generation from patients' peripheral blood cells, and that the in vitro differentiated platform of FC-iPSC-CM is highly representative, both genotypically and phenotypically, of the disease phenotypic of Fabry-specific cardiomyopathy.

2. Upregulation of Cardiac Alox12/15 and Secretory 12(S)-HETE/15(S)-HETE in FC-iPSC-CMs and Clinical Samples of Fabry Patients with Cardiomyopathy

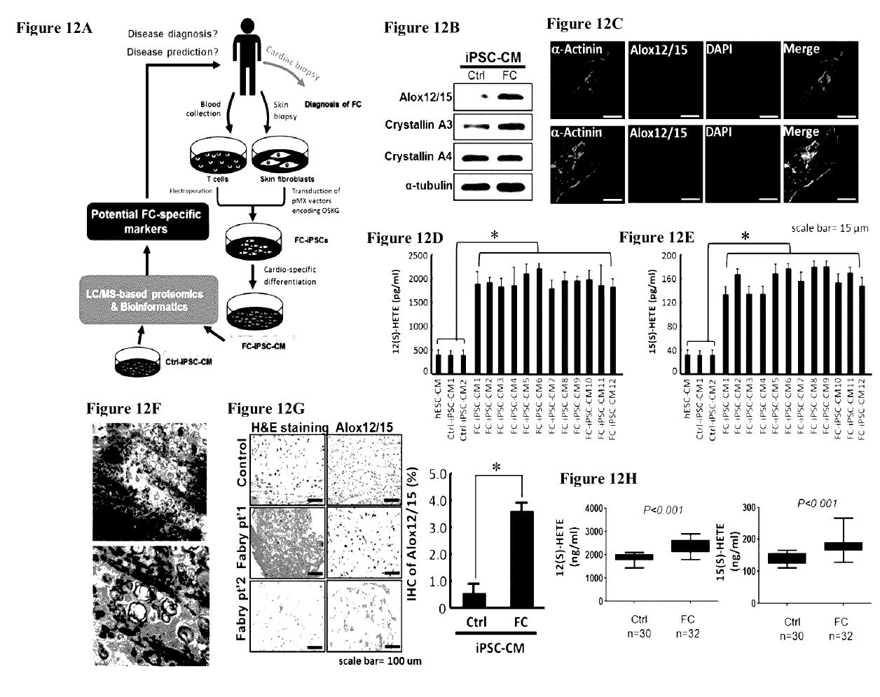

The recapitulation of FC-specific characteristics in our FC-iPSC-CMs indicated that FC-iPSC-CMs may represent a useful platform for investigating the pathogenesis of FC and screening of potential FC-specific biomarkers. Here, we used liquid chromatography-mass spectrometry-based proteomic analysis (LC/MS) and bioinformatics to screen for suitable markers of FC in our FC-iPSC-CM platform (FIG. 12A). Among the identified potential markers, arachidonate lipoxygenases 12/15 (Alox12/15) were the most highly upregulated in FC-iPSC-CMs compared with Ctrl-iPSC-CMs (Table 9). This result was also confirmed by Western blot (FIG. 12B) and immunofluorescence (FIG. 12C). Enzyme-linked immunosorbent assay (ELISA) analysis revealed that the secretion of Alox12/15 metabolites, 12(S)-HETE and 15(S)-HETE, were increased in the medium of FC-iPSC-CMs (FIG. 12D and 12E). Both Alox12/15 and 12(S)-HETE/15(S)-HETE were increased in a time-dependent manner after myocardial differentiation.

TABLE 9
Proteomic Data of FC-iPSC-CMs as compared with control-iPSC-CMs

| | | | Gene | | Unique | MW | |
|---|---|---|---|---|---|---|---|
| No. | Protein Name | Uniprot | Name | F/C | # | [kDa] | PEP |
| 1 | Arachidonate 15-lipoxygenase | P16050 | ALOX15 | ∞ | 4 | 77.48 | 1.49E−07 |
| 2 | Beta-crystallin A3 | P05813 | CRYBA1 | ∞ | 1 | 25.15 | 3.41E−48 |
| 3 | Beta-crystallin A4 | P53673 | CRYBA4 | ∞ | 5 | 22.37 | 3.31E−70 |
| 6 | Netrin receptor DCC | P43146 | DCC | ∞ | 2 | 158.45 | 2.00E−13 |
| 7 | Ribosyldihydronicotinamide dehydrogenase [quinone] | P16083 | NQO2 | ∞ | 4 | 25.92 | 6.86E−09 |
| 8 | NAD(P)H dehydrogenase [quinone] 1 | P15559 | NQO1 | ∞ | 3 | 30.87 | 1.52E−04 |
| 9 | Coiled-coil domain-containing protein 58 | Q4VC31 | CCDC58 | ∞ | 3 | 16.62 | 6.24E−08 |
| 10 | Phosphoglucomutase-like protein 5 | Q15124 | PGM5 | ∞ | 4 | 62.22 | 2.79E−25 |
| 11 | tRNA-splicing endonuclease subunit Sen54 | Q7Z6J9 | TSEN54 | ∞ | 2 | 58.82 | 2.82E−02 |
| 12 | Lamin-B receptor | Q14739 | LBR | ∞ | 4 | 70.70 | 9.64E−16 |
| 13 | Ribulose-phosphate 3-epimerase | Q96AT9 | RPE | ∞ | 2 | 24.93 | 1.01E−42 |
| 14 | Neuronal membrane glycoprotein M6-a | P51674 | GPM6A | ∞ | 4 | 31.21 | 4.89E−05 |
| 15 | Syndecan-2 | P34741 | SDC2 | ∞ | 2 | 22.16 | 3.89E−05 |
| 16 | Mitochondrial import inner membrane translocase | O60830 | TIMM17B | ∞ | 2 | 23.82 | 6.33E−05 |
| | subunit Tim17-B | | | | | | |
| 17 | Neuropilin-1 | O14786 | NRP1 | ∞ | 3 | 103.13 | 7.54E−07 |
| 18 | UPF0552 protein C15orf38 | E2QRD5 | C15orf38 | ∞ | 2 | 43.88 | 1.45E−03 |
| 19 | PEST proteolytic signal-containing nuclear protein | Q8WW12 | PCNP | ∞ | 2 | 20.89 | 1.90E−03 |
| 20 | Glutaminase kidney isoform, mitochondrial | O94925 | GLS | ∞ | 2 | 73.46 | 6.43E−104 |

To further investigate the role of cardiac Alox12/15 in the pathogenesis of FC, thirty-two FC patients with left ventricular hypertrophy (LVH) initially diagnosed by echography and the phenomenon of Gb3 deposition in the myocardial biopsied samples (FIG. 12F) were enrolled in this study. These FC patients had not previously received enzyme replacement therapy (ERT).

Immunohistochemistry results indicated that a remarkable number of Alox12/15-positive cells were detected in the 7 cardiac biopsied samples compared with normal heart sample control (Heart Test Tissue Array T301, provided by US Biomax Inc., Rockville, Md.; FIG. 12G). Consistently, increased serum levels of 12(S)-HETE (2323±46.2 ng/mL vs. 1843±21.6 ng/mL, P<0.001) and 15(S)-HETE (181.1±4.4 ng/mL vs. 141.9±2.4 ng/mL, P<0.001; FIG. 12H) were observed in the 32 FC patients who had LVH without ERT. Collectively, our observations indicated that cardiac Alox12/15 and its metabolites 12(S)-HETE and 15(S)-HETE might be involved in the pathogenesis of Fabry-associated cardiomyopathy.

3. Significant Correlation of Fibrotic Markers with Cardiac Alox12/15 and Secreted 12(S)-HETE/15(S)-HETE in Late-Differentiation Stage of FC-iPSC-CMs

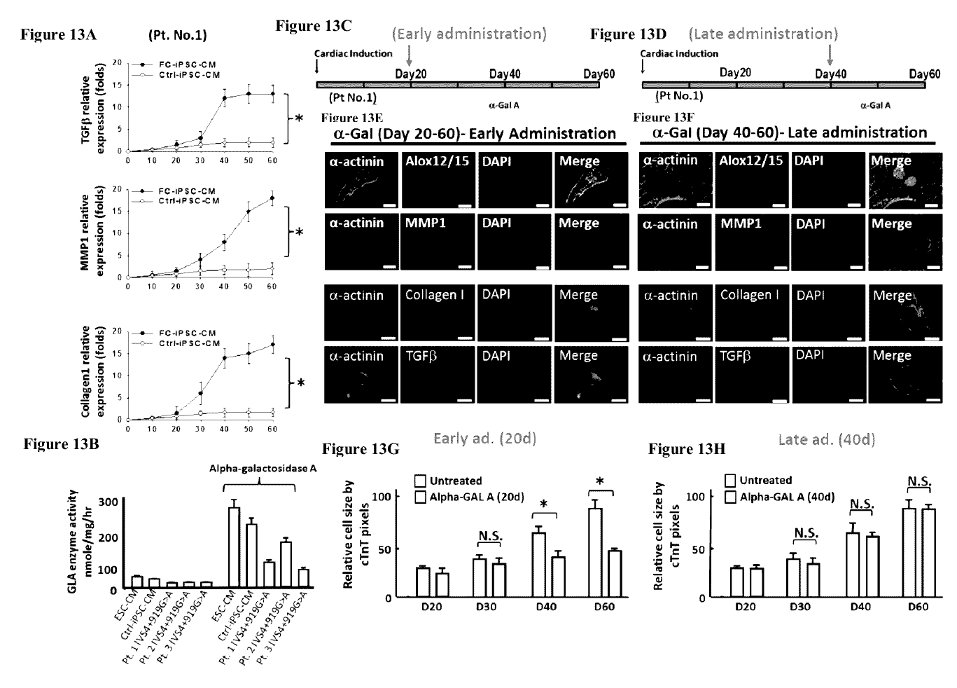

Myocardial fibrosis in FD is a progressive process that is apparently not modified by ERT and is a crucial outcome determinant. Our TEM and other results have demonstrated FC-iPSC-CMs as a feasible platform that models FC-associated cardiac manifestations at day 40 to 60 post-induction (FIG. 11). We further observed an upregulation of fibrotic markers (collagen1, TGFβ, and MMP1) from day 40 post-differentiation, which reached the maximal expression at day 60 post-differentiation in FC-iPSC-CMs (FIG. 13A: Patient 1 with IVS4 +919G >A mutation). The late upregulation of fibrotic markers is in line with the feature of late onset of myocardial fibrosis in FC. Subsequently, we examined the treatment response of ERT in the FC-iPSC-CM platform (administration during day 40-60 post-differentiation). The GLA activity in FC-iPSC-CMs was consistently reduced compared with that in ESC-derived cardiomyocytes (ESC-CMs) or Ctrl-iPSC-CMs. At day 20 post-induction, though the administration of GLA (5 μg/ml for 5 days) elicited high GLA activity among all experimental cells, the GLA activity in FC-iPSC-CMs was still comparatively lower than in Ctrl-iPSC-CMs (FIG. 13B).

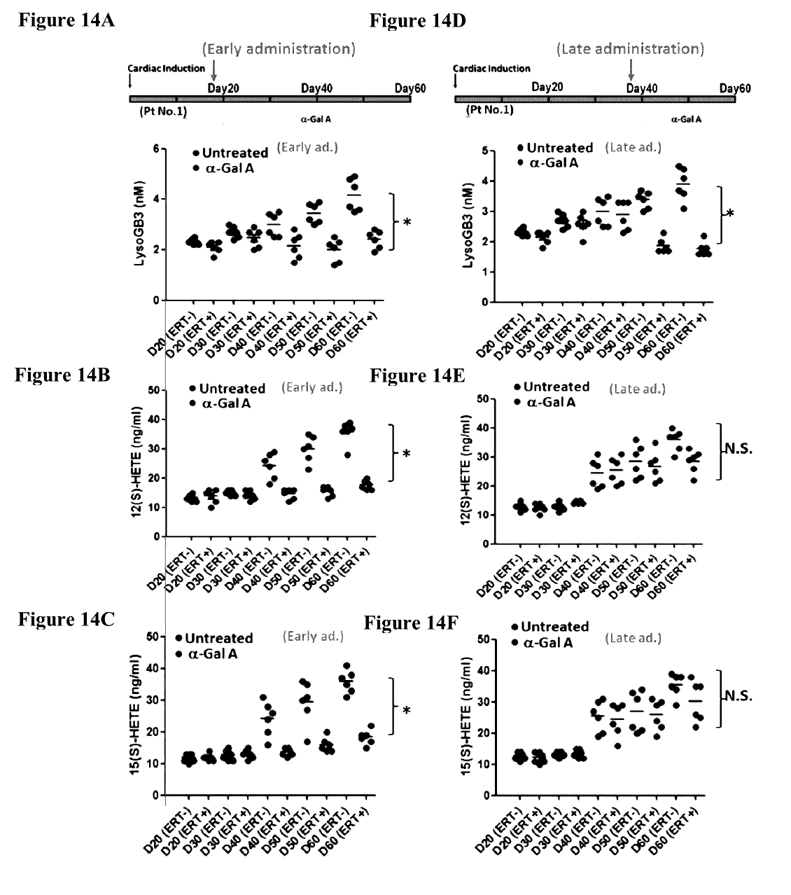

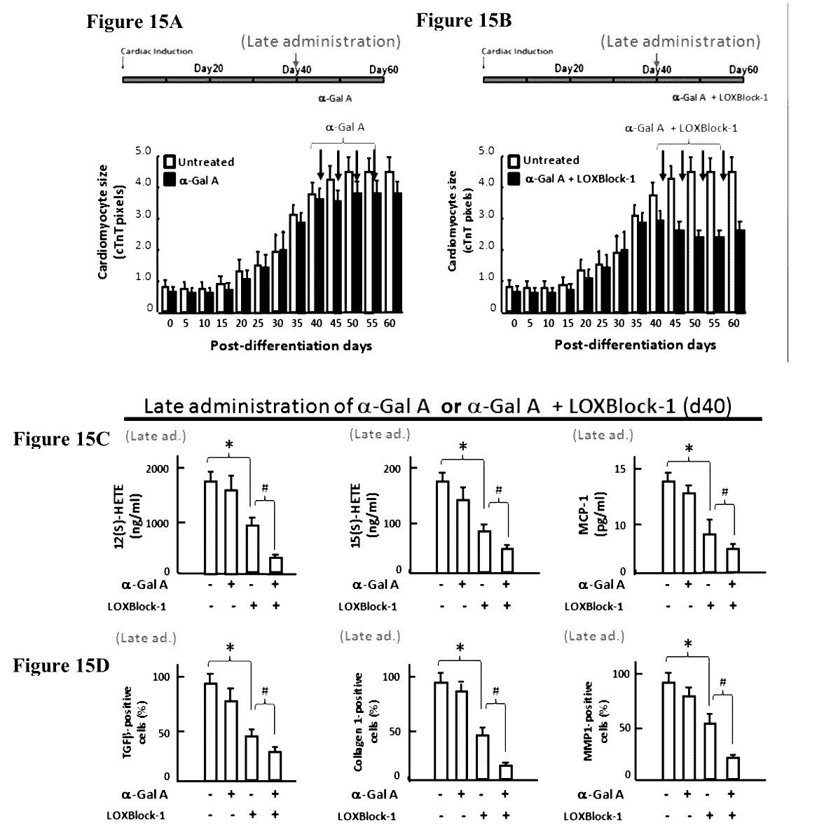

ERT exhibits better efficacy before the development of myocardial fibrosis in FC. However, the interrelationship between treatment onset and the efficacy of ERT drugs in FC with fibrotic progression remains unclear. Therefore, we compared the treatment efficacy of GLA between early (5 μg/ml of GLA from day 20 to day 60 post-differentiation; FIG. 13C) and late (5 μg/ml of GLA from day 40 to day 60 post-differentiation; FIG. 13D) administrations in FC-iPSC-CMs, and evaluated the levels of cardiac Alox12/15 and fibrotic markers at day 60 post-differentiation. First, the immunofluorescent results indicated that early-administration of GLA suppressed the expression levels of Alox12/15 as well as fibrotic biomarkers MMP1, TGF-β and collagen1 in FC-iPSC-CMs to a larger extent than late-administration (FIG. 13E and 13F). Second, quantification of c-TnT-positive margins indicated that the cell size of FC-iPSC-CMs was prominently reduced by early-administration of GLA (FIG. 13G), but not late-administration (FIG. 13H). Finally, compared with untreated cells, early-administration of GLA significantly suppressed the expression of fibrotic proteins as well as the secreted levels of LyosGB3 (FIG. 14A), 12-HETE (FIG. 14B), and 15-HETE (FIG. 14C) at day 60 of FC-iPSC-CMs. In contrast, late-administration of GLA resulted in a negligible inhibitory effect on fibrotic protein expression and the secretion of 12(S)-HETE (FIG. 14E) and 15(S)-HETE (FIG. 14F) despite a significant reduction in lysoGb3 (FIG. 14D), indicating that 12(S)-HETE and 15(S)-HETE correlated better than lysoGb3 with myocardial-fibrotic changes in FC-iPSC-CMs at day 60 post-differentiation. Additionally, recent studies suggested the Alox12/15 pathway as a therapeutic target in focal ischemia and cardiac inflammation. We therefore tested the treatment efficacy of the pharmacological inhibition of cardiac Alox12/15 in combination with ERT (FIG. 15). Compared with late-administration of GLA alone, the addition of 2 μM LOXBlock-1 (ChemBridge, San Diego, Calif.), an Alox12/15 inhibitor, potently enhanced the effect of late-administrated GLA and reduced the cardiomyocyte size, secreted levels of 12(S)-HETE and 15(S)-HETE, and the expression levels of fibrotic markers (MMP1, TGF-β and collagen1) in FC-iPSC-CMs after 60 days of differentiation. These results suggested that LOXBlock-1 exhibited a synergistic efficacy with ERT that potently inhibited the myocardial-fibrosis in late-differentiation stage of FC-iPSC-CMs. Collectively, our data indicated that cardiac Alox12/15 and secreted 12(S)-HETE/15(S)-HETE significantly correlated with the ERT response in the fibrotic progression of FC-iPSC-CMs.

### 4. ALox12/15 and Secreted 12(S)-HETE/15(S)-HETE but not LysoGb3 Reflected Progressive Myocardial Fibrosis and LV Systolic Function in FC Patients

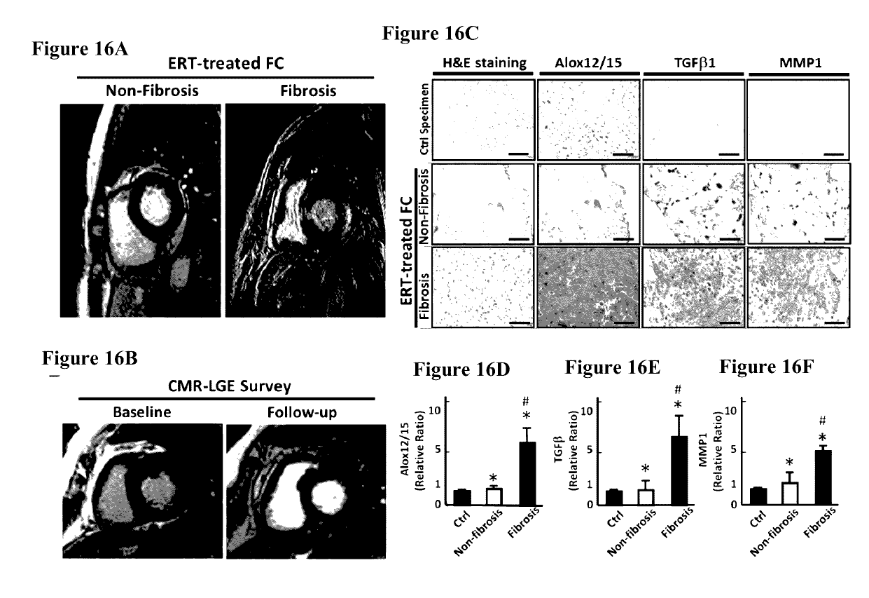

To further explore whether cardiac Alox12/15 and secreted 12(S)-HETE/15(S)-HETE play a vital role in progressive myocardial-fibrosis of FC patients, CMR-LGE was used to validate the development of myocardial-fibrosis in ERT treated FC patients (FIG. 16A and 16B). We enrolled 47 FC patients who received 1 to 3.6 years of ERT (mean F/U time: 2.2±1.1 years; range: 1.0-3.6 years) for the study. Table 6 lists the demographic and clinical characteristics, as well as mean LV mass, volume and ejection fraction of the 47 patients. As shown in Table 6, among the 17 FC patients with myocardial fibrosis identified by CMR-LGE (FIG. 14A), the serum levels of 12(S)-HETE and 15(S)-HETE were significantly higher than that in the other 30 patients without myocardial fibrosis. However, there was no detectable differences in lysoGb3 levels between these two FC cohorts (R²=0.862 and 0.909 for 12(S)- and 15(S)-HETE, respectively, both P<0.05; Table 1A). Furthermore, the immunohistochemical results of the cardiac-biopsied samples revealed that the expression levels of Alox12/15 and fibrotic markers (TGFβ and MMP1) were significantly increased in FC-patients with myocardial fibrosis compared with those without myocardial fibrosis (FIG. 16C through 16F), whereas these markers (Alox12/15, TGFβ, and MMP1) were barely detectable in normal-control samples (FIG. 16C through 16F). Notably, compared between normal-control, non-fibrosis FC, and fibrosis FC, the cardiac expression levels of Alox12/15 highly paralleled the expression levels of TGFβ and MMP1 (FIG. 16D through 16F). In consistent with our in vitro observations in FC-iPSC-CMs, our data supported that high levels of cardiac Alox12/15 expression paralleled with increased fibrotic markers as well as the insensitiveness to ERT-response in FC-with myocardial fibrosis. Moreover, the increase of secreted 12(S)-HETE and 15(S)-HETE, the downstream effectors of Alox12/15, in FC-patients suggested that 12(S)-HETE and 15(S)-HETE may potentially the surrogate biomarkers reflecting the myocardial-fibrotic progression in ERT-treated FC.

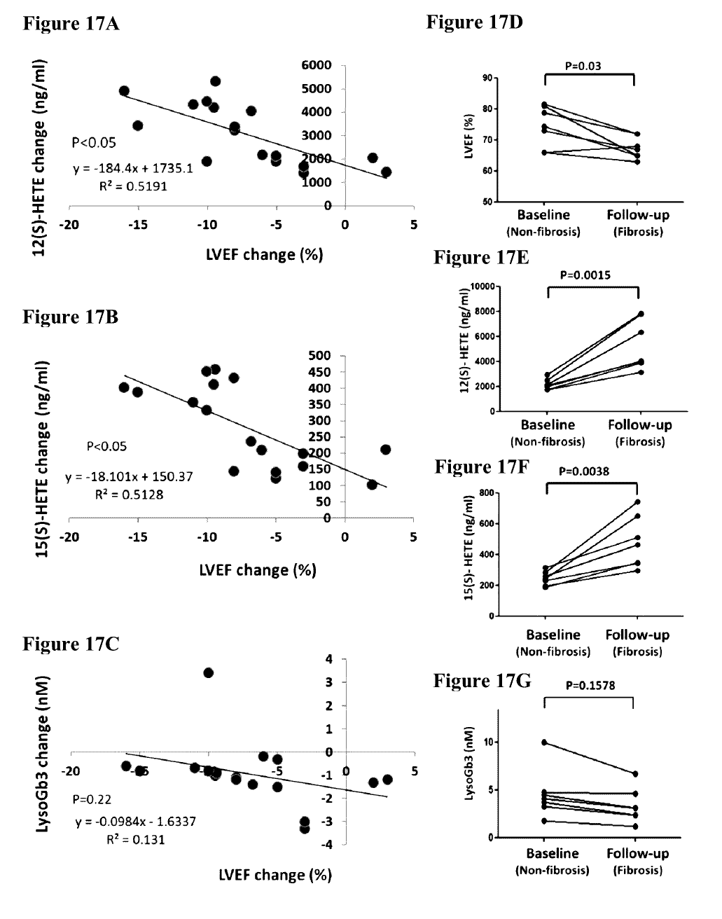

Alox12/15 and 12(S)-HETE/15(S)-HETE induce systolic dysfunction and heart failure in transgenic animals. We explored the impact of myocardial fibrosis on cardiac dysfunction using CMR-LGE imaging and biomarker evaluation to determine the progression of FC-patients under ERT. The consecutive follow-up information, including global LV function and other clinical parameters of FC-patients with myocardial fibrosis, showed the progression on repeat CMR-LGE (FIG. 16B). The changes in the magnitudes of LVEF, secreted 12(S)-HETE/15(S)-HETE, and lysoGB3 in the 17 FC-patients with myocardial-fibrosis under ERT were further evaluated (Table 6). Our results revealed that the changes in LVEF (• LVEF) negatively correlated with the change in serum level of 12(S)-HETE and 15(S)-HETE [• 12(S) and • 15(S)-HETE] (FIG. 17A and 17B) in these 17 FC-patients; however, no statistically significant correlation was observed between • LVEF and • lysoGB3 (FIG. 17C) in these 17 FC patients under ERT. Furthermore, 7 out of the 17 FC-patients with myocardial fibrosis initially exhibited no myocardial fibrosis at baseline but subsequently developed myocardial-fibrosis during follow-up. In these 7 newly developed FC-patients with myocardial-fibrosis, the secretion of 12(S)-HETE/15(S)-HETE (FIG. 17E and 17F), but not LysoGB3 (FIG. 17G), dramatically elevated along with decreased LVEF function diagnosis by CMR-LGE (FIG. 17D). No correlation was detected between the levels of serum lysoGB3 and the changes of systolic LVEF function, signifying the limitation of lysoGb3 for prognostic implications under ERT. The highly association between the changes in the magnitudes of secreted 12(S)-HETE/15(S)-HETE and cardiac dysfunction (FIG. 17) suggested that serum levels of 12(S)-HETE/15(S)-HETE may reflect the systolic LV-dysfunction better than LysoGb3 during the occurrence of myocardial-fibrosis in ERT-treated FC. Altogether, serum 12(S)- and 15(S)-HETE could be used as prognostic biomarkers for evaluating the ERT-therapeutic responses, including systolic LV-function and myocardial-fibrotic progression, in patients with Fabry-associated cardiomyopathy.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for preparing induced pluripotent stem cells (iPSCs) from somatic cells, comprising:
(a) transfecting or transducing the transcription factor into isolated somatic cells, or contacting or exposing isolated somatic cells with/to transcription factor, which the isolated somatic cells can express transcription factor; and
(b) culturing the isolated somatic cells as obtained in step (a) under appropriate conditions, thereby converting the somatic cells into iPSCs and maintaining pluripotency and self-renewal ability,
wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

2. The method of claim 1, wherein the isolated somatic cells are transfected or transduced with one or more plasmid or vector comprising transcription factor operably linked to a promoter, wherein the transcription factor is selected from the group consisting of Oct-3/4, Sox2, Klf4, Glial, Parp1, ASH2L, c-Myc, Lin28, Nanog, Rex1, DPPA2, DPPA4, DPPA5, GDF3, SSEA3, SSEA4, Tra-1-60, Tra-1-81 and combination thereof.

3. The method of claim 2, wherein the vector is a viral vector.

4. The method of claim 2, wherein the isolated somatic cells are transfected by electroporation.

5. The method of claim 1, wherein the transcription factors are Oct-4, Sox2, Klf4, and Glial.

6. The method of claim 1, wherein the isolated somatic cells are fibroblasts, nerve cells, amniotic fluid cells, bone marrow cells, blood cells, myocardial cells, dermal or epidermal cells, connective tissue cells, chondrocytes, rod and cone cells, retinal pigment epithelia, or pancreatic cells.

7. The method of claim 6, wherein the fibroblast is dermal fibroblast.

8. The method of claim 6, wherein the blood cell is peripheral blood mononuclear cell.

9. The method of claim 1, wherein the iPSCs can differentiate to nervous system, teeth, hair, exocrine glands, epithelium, or mesenchyme from ectoderm.

10. The method of claim 1, wherein the iPSCs can differentiate to the muscle of smooth, cardiac and skeletal, the muscles of the tongue, the pharyngeal arches muscle, connective tissue, dermis and subcutaneous layer of the skin, bone and cartilage, dura mater, endothelium of blood vessels, red blood cells, white blood cells, microglia and Kupffer cells, the kidneys and the adrenal cortex cartilage, gonads, or keratinocytes from mesoderm.

11. The method of claim 1, wherein the iPSCs can differentiate to lung cells, thyroid cells, pancreatic cells, liver cells, retinal pigment epithelium, or eyes from endoderm.

12. An iPSC(s) obtained by the method of claim 1.

13. A process of drug selection for the treatment of a heritable genetic disease, comprising the steps of:
(1) isolating the somatic cells from a subject with a heritable genetic disease,
(2) preparing the iPSCs as the method of claim 1,
(3) differentiating the iPSCs obtained from step (2) into a specific cell line having the gene of the heritable genetic disease and affected by the disease, and
(4) selecting a drug for improving the condition of the cells of the cell line affected by the heritable genetic disease.

14. The process of claim 13, wherein the heritable genetic disease is selected from the group consisting of Fabry disease, cystic fibrosis, sickle-cell anemia, polydactyly, Huntingdon's disease, ALA dehydratase deficiency, aceruloplasminemia, achondroplasia, Turner syndrome, Down syndrome, Klinefelter syndrome, Gaucher disease type 1 and type 2, Apert syndrome, Pfeiffer syndrome, acute intermittent porphyria, Canavan disease, Alzheimer's disease, and Muenke syndrome.

15. A process of drug selection for Fabry disease, comprising the steps of:
(1) isolating the somatic cells from a subject with Fabry disease,
(2) preparing the iPSCs as the method of claim 1,
(3) differentiating the iPSCs obtained from step (2) into hypertrophic cardiomyocytes having Fabry disease and affected by the disease,
(4) selecting a drug for improving the condition of the hypertrophic cardiomyocytes affected by Fabry disease.

16. The process of claim 15, wherein the hypertrophic cardiomyocytes detected by one or more biomarkers selected from the group consisting of Alox12, Alox15, 12-HETE, and 15-HETE, which have a high level in one or more of the biomarkers as compared to normal cardiomyocytes.

17. A method for treating Fabry-associated myocardiopathy in a subject in need thereof, comprising administering to the subject an effective amount of an arachidonate lipoxygenases 12/15 (Alox 12/15) inhibitor.

18. The method of claim 17, wherein the Alox 12/15 inhibitor is selected from the group consisting of LOXBlock-1, LOXBlock-2, LOXBlock-3 and a combination thereof.

19. The method of claim 18, wherein the Alox 12/15 inhibitor is LOXBlock-1.

20. The method of claim 17, wherein the Alox 12/15 inhibitor is administered simultaneously with an effective amount of α-galactosidase A (GLA).

21. A method for determining whether a subject has poor prognosis for Fabry-associated myocardiopathy, comprising:
extracting a test sample of tissue from a subject;
measuring the level of Alox 12/15 or 12(S)-HETE/15(S)-HETE in the test sample; and
determining the prognosis of the subject, wherein an alteration in the levels of Alox 12/15 or 12(S)-HETE/15(S)-HETE level in the test sample, relative to the corresponding Alox 12/15 or 12(S)-HETE/15(S)-HETE level in a control sample of Fabry-disease free tissue, is indicative of the subject having a poor prognosis for Fabry-associated myocardiopathy.

22. The method of claim 21, wherein the poor prognosis for Fabry-associated myocardipathy is manifested as a syndrome selected from the group consisting of myocardial fibrosis, decreased systolic left ventricular (LV) function and a combination thereof.

23. The method of claim 21, wherein the subject has received an enzyme replacement therapy (ERT).


SEQUENCE LISTING (26 sequences)

SEQ ID NO: 1 - DNA, 21 bp, Homo sapiens
cttcaggcactgtgttcattg

SEQ ID NO: 2 - DNA, 20 bp, Homo sapiens
tttggctgaacaccttccca

SEQ ID NO: 3 - DNA, 20 bp, Homo sapiens
gccctgcagtacaactccat

SEQ ID NO: 4 - DNA, 20 bp, Homo sapiens
ttcctgcaaagctcctaccg

SEQ ID NO: 5 - DNA, 20 bp, Homo sapiens
gaagacaaggtcccggtcaa

SEQ ID NO: 6 - DNA, 20 bp, Homo sapiens
ggattcagccagtgtccaga

SEQ ID NO: 7 - DNA, 20 bp, Homo sapiens
gtgggccttatgtgatggct

SEQ ID NO: 8 - DNA, 20 bp, Homo sapiens
tgcgttaggatgtgggcttt

SEQ ID NO: 9 - DNA, 24 bp, Homo sapiens
ccgtccccgcaatctccttccatc

SEQ ID NO: 10 - DNA, 24 bp, Homo sapiens
atgatgccaacatggctcccggtg

SEQ ID NO: 11 - DNA, 20 bp, Homo sapiens
tagcacagcaaaagaggcca

SEQ ID NO: 12 - DNA, 20 bp, Homo sapiens
tgcatggcccataaacaggt

SEQ ID NO: 13 - DNA, 20 bp, Homo sapiens
cggcacgtagacatatcccg

SEQ ID NO: 14 - DNA, 20 bp, Homo sapiens
ggcttcattgcattggctgg

SEQ ID NO: 15 - DNA, 20 bp, Homo sapiens
gtttgtgttgcggtcagtcc

SEQ ID NO: 16 - DNA, 20 bp, Homo sapiens
ttggtggggatacacacagc

SEQ ID NO: 17 - DNA, 20 bp, Homo sapiens
gtcgccagccgagccacatc

SEQ ID NO: 18 - DNA, 20 bp, Homo sapiens
ccaggcgcccaatacgacca

SEQ ID NO: 19 - DNA, 20 bp, Homo sapiens
gcctagggacagactgcaag

SEQ ID NO: 20 - DNA, 20 bp, Homo sapiens
ggcgaggaagtcaccatcaa

SEQ ID NO: 21 - DNA, 18 bp, Homo sapiens
cagggcgtcatggtcggt

SEQ ID NO: 22 - DNA, 24 bp, Homo sapiens
gtgccagatcttctccatgtcatc

SEQ ID NO: 23 - DNA, 20 bp, Homo sapiens
ctcatctctctcccccgagt

SEQ ID NO: 24 - DNA, 20 bp, Homo sapiens
tggaacatggcctctggatg

SEQ ID NO: 25 - DNA, 22 bp, Homo sapiens
ggagttcaaagaagccttcagc

SEQ ID NO: 26 - DNA, 20 bp, Homo sapiens
ctcctctgggacactcacct